(19) United States
(12) Patent Application Publication
(10) Pub. No.: US 2022/0226100 A1
(43) Pub. Date: Jul. 21, 2022

(54) COMPRESSION ANCHOR SYSTEMS, DEVICES, INSTRUMENTS, IMPLANTS AND METHODS OF ASSEMBLY AND USE

(71) Applicant: RAFAEL HOLDINGS, INC., Newark, NJ (US)

(72) Inventor: Gavriel FEUER, Montclair, NJ (US)

(73) Assignee: RAFAEL HOLDINGS, INC., Newark, NJ (US)

(21) Appl. No.: 17/657,873

(22) Filed: Apr. 4, 2022

Related U.S. Application Data
(63) Continuation of application No. PCT/US2022/012505, filed on Jan. 14, 2022.
(60) Provisional application No. 63/199,678, filed on Jan. 15, 2021.

(51) Int. Cl. A61F 2/08; A61B 17/88; A61B 17/86

(52) U.S. Cl. CPC A61F 2/0811; A61B 17/8875; A61B 2017/681; A61F 2002/0852; A61B 17/863

(57) ABSTRACT

Compression anchor systems, devices, instruments, implants, and methods for using the compression anchor systems, devices, instruments, and implants are disclosed. The compression anchor system including an implant and a suture anchor. The compression anchor system may also include an inserter for engaging the implant and receiving at least a portion of the suture anchor. The implant may include a threaded head and a shaft with at least one thread portion and at least one anchor portion. Finally, methods for assembling and surgically inserting the anchor system are also disclosed.

### CROSS REFERENCE TO RELATED APPLICATION

This application is a continuation of PCT Application No. PCT/US2022/012505 filed Jan. 14, 2022 and entitled Compression Anchor Systems, Devices, Instruments, Implants and Methods of Assembly and Use, which claims priority benefit under 35 U.S.C. § 119(e) of U.S. Provisional Application No. 63/199,678 filed Jan. 15, 2021 and entitled Compression Anchor Systems, Devices, Instruments, Implants and Methods of Assembly and Use (Attorney Docket No. 5940.002P1), which are incorporated herein by reference in their entireties.

### FIELD OF THE INVENTION

The present invention relates generally to general surgery, orthopaedic implants used for fracture compression and soft tissue anchoring. More specifically, but not exclusively, the present invention relates to compression anchor systems, devices, instruments, implants, and methods for assembling and using the compression anchor systems, devices, instruments, and implants.

### BACKGROUND OF THE INVENTION

Often when there is a fracture dislocation or any bone fracture the surrounding ligaments, tendons or other soft tissue may also be injured. Some current methods for repairing these hard and soft tissue injuries include using separate implants or devices to repair the hard tissue or bone injuries and the soft tissue, tendon or ligament injuries. Other methods include a combination implant or device for addressing both the hard and soft tissue injuries. However, the currently available combination implants may include complex components or structures which increase the probability for failure of the sutures. The suture failures may include suture breakage, suture slippage, or the like failures which would be understood by one of ordinary skill in the art. Thus, improved anchoring mechanisms and structures are needed for bone screws to prevent suture failures and to improve the ability to stabilize a fracture dislocation or any bone fracture while also allowing for soft tissue repair of surrounding or associated soft tissue injuries. In addition, improved inserters are needed to address the free ends of the sutures during insertion of the bone screws to avoid suture damage during implantation.

### SUMMARY OF THE INVENTION

Aspects of the present invention provide compression anchor systems, devices, instruments, and implants, and methods for assembling and using the compression anchor systems, devices, instruments, and implants for fusing bones or bone pieces and anchoring soft tissue.

In one aspect, provided herein is an anchor system including an implant and a suture coupled to at least a portion of the implant.

In another aspect, provided herein is an implant including a head at a first end of the implant, a shaft coupled to and extending from the head to a second end of the implant, and a cannulation extending through the implant from the first end to the second end. The shaft including at least one second threaded section, a smooth section positioned adjacent to the at least one second threaded section, and at least one anchor portion positioned on the smooth section of the shaft. The anchor portion including a first opening extending through the shaft and into the cannulation, a second opening extending through the shaft and into the cannulation, wherein the second opening is spaced apart from the first opening, and a first recess inset into an exterior surface of the implant, wherein the first recess extends between the first opening and the second opening.

In yet another aspect, provided herein is an inserter including a handle extending from a first end of the inserter toward a second end and a drive portion extending from the handle to the second end of the inserter.

In still another aspect, provided herein is a method of assembling a compression anchor system.

In a further aspect, provided herein is a surgical method including obtaining an orthopaedic compression anchor and preparing the bones around a fracture site. The method also includes stabilizing the fracture and determining the length of the implant needed. In addition, the method includes inserting a fixation device across the fracture and using imaging to verify the position of the fixation device. The method also includes preloading the implant with the suture and inserting the compression anchor across the fracture. In addition, the method may include removing the inserter from the compression anchor and releasing the sutures and performing the soft tissue repair with the sutures. Further, the method includes completing the procedure.

These, and other objects, features and advantages of this invention will become apparent from the following detailed description of the various aspects of the invention taken in conjunction with the accompanying drawings.

### BRIEF DESCRIPTION OF DRAWINGS

The accompanying drawings, which are incorporated in and constitute a part of the specification, illustrate embodiments of the invention and together with the detailed description herein, serve to explain the principles of the invention. The drawings are only for purposes of illustrating preferred embodiments and are not to be construed as limiting the invention. It is emphasized that, in accordance with the standard practice in the industry, various features are not drawn to scale. In fact, the dimensions of the various features may be arbitrarily increased or reduced for clarity of discussion. The foregoing and other objects, features and advantages of the invention are apparent from the following detailed description taken in conjunction with the accompanying drawings in which:

FIG. 1 is a perspective view of one embodiment of an orthopaedic compression anchor system with a transparent implant, in accordance with an aspect of the present disclosure;

FIG. 2 is another perspective view of the compression anchor system of FIG. 1 with a transparent inserter and implant, in accordance with an aspect of the present disclosure;

FIG. 3 is an exploded, first perspective view of the compression anchor system of FIG. 1, in accordance with an aspect of the present disclosure;

FIG. 4 is an exploded, second perspective view of the compression anchor system of FIG. 1, in accordance with an aspect of the present disclosure;

FIG. 5 is a first perspective view of the implant of the compression anchor system of FIG. 1, in accordance with an aspect of the present disclosure;

FIG. 6 is a second perspective view of the implant of FIG. 5, in accordance with an aspect of the present disclosure;

FIG. 7 is a first side view of the implant of FIG. 5, in accordance with an aspect of the present disclosure;

FIG. 8 is a second side view of the implant of FIG. 5, in accordance with an aspect of the present disclosure;

FIG. 9 is a third side view of the implant of FIG. 5, in accordance with an aspect of the present disclosure;

FIG. 10 is a fourth side view of the implant of FIG. 5, in accordance with an aspect of the present disclosure;

FIG. 11 is a first end view of the implant of FIG. 5, in accordance with an aspect of the present disclosure;

FIG. 12 is a second end view of the implant of FIG. 5, in accordance with an aspect of the present disclosure;

FIG. 13 is a first cross-sectional side perspective view of the implant of FIG. 5 taken along line 13-13 in FIG. 9, in accordance with an aspect of the present disclosure;

FIG. 14 is a second cross-sectional side perspective view of the implant of FIG. 5 taken along line 14-14 in FIG. 7, in accordance with an aspect of the present disclosure;

FIG. 15 is a perspective view of the assembled implant and suture anchor of FIG. 1 with a transparent implant, in accordance with an aspect of the present disclosure;

FIG. 16 is a cross-sectional side view of the assembled implant and suture anchor of FIG. 15 taken along line 16-16 in FIG. 15, in accordance with an aspect of the present disclosure;

FIG. 17 is a side view of the inserter of the compression anchor system of FIG. 1, in accordance with an aspect of the present disclosure;

FIG. 18 is a top and/or bottom view of the inserter of FIG. 17, in accordance with an aspect of the present disclosure;

FIG. 19 is a first cross-sectional side view of the inserter of FIG. 17 taken along line 19-19 in FIG. 17, in accordance with an aspect of the present disclosure;

FIG. 20 is a second cross-sectional side view of the inserter of FIG. 17 taken along line 20-20 in FIG. 18, in accordance with an aspect of the present disclosure;

FIG. 21 is a perspective view of another embodiment of an implant and suture anchor for an orthopaedic compression anchor system, in accordance with an aspect of the present disclosure;

FIG. 22 is another perspective view of the implant and suture anchor of FIG. 21 with a transparent implant, in accordance with an aspect of the present disclosure;

FIG. 23 is a first, perspective view of the implant of FIG. 21, in accordance with an aspect of the present disclosure;

FIG. 24 is a second, perspective view of the implant of FIG. 21, in accordance with an aspect of the present disclosure;

FIG. 25 is a first side view of the implant of FIG. 21, in accordance with an aspect of the present disclosure;

FIG. 26 is a second side view of the implant of FIG. 21, in accordance with an aspect of the present disclosure;

FIG. 27 is a third side view of the implant of FIG. 21, in accordance with an aspect of the present disclosure;

FIG. 28 is a fourth side view of the implant of FIG. 21, in accordance with an aspect of the present disclosure;

FIG. 29 is a first end view of the implant of FIG. 21, in accordance with an aspect of the present disclosure;

FIG. 30 is a second end view of the implant of FIG. 21, in accordance with an aspect of the present disclosure;

FIG. 31 is a first cross-sectional view of the implant of FIG. 21 taken along line 31-31 in FIG. 26, in accordance with an aspect of the present disclosure;

FIG. 32 is a second cross-sectional view of the implant of FIG. 21 taken along line 32-32 in FIG. 25, in accordance with an aspect of the present disclosure;

FIG. 33 is a method of inserting an orthopaedic compression anchor into a patient's bone(s) and securing the suture anchor to a patient's soft tissue, in accordance with an aspect of the present disclosure;

FIG. 34 is a perspective view of another embodiment of an orthopaedic compression anchor system, in accordance with an aspect of the present disclosure;

FIG. 35 is a top view of the compression anchor system of FIG. 34, in accordance with an aspect of the present disclosure;

FIG. 36 is a side view of the compression anchor system of FIG. 34, in accordance with an aspect of the present disclosure;

FIG. 37 is an exploded, first perspective view of the compression anchor system of FIG. 34, in accordance with an aspect of the present disclosure;

FIG. 38 is an exploded, second perspective view of the compression anchor system of FIG. 34, in accordance with an aspect of the present disclosure;

FIG. 39 is a top view of an orthopaedic compression anchor of the system of FIG. 34 with a transparent implant, in accordance with an aspect of the present disclosure;

FIG. 40 is a perspective view of the orthopaedic compression anchor of FIG. 39, in accordance with an aspect of the present disclosure;

FIG. 41 is a magnified view of a portion of the orthopaedic compression anchor of FIG. 39, in accordance with an aspect of the present disclosure;

FIG. 42 is a cross-sectional view of a portion of the orthopaedic compression anchor of FIG. 39 taken along line 42-42 in FIG. 39, in accordance with an aspect of the present disclosure;

FIG. 43 is a cross-sectional view of a portion of the orthopaedic compression anchor of FIG. 39 taken along line 43-43 in FIG. 39, in accordance with an aspect of the present disclosure;

FIG. 44 is a first perspective view of the implant of the orthopaedic compression anchor system of FIG. 34, in accordance with an aspect of the present disclosure;

FIG. 45 is a second perspective view of the implant of FIG. 44, in accordance with an aspect of the present disclosure;

FIG. 46 is a first side view of the implant of FIG. 44, in accordance with an aspect of the present disclosure;

FIG. 47 is a second side view of the implant of FIG. 44, in accordance with an aspect of the present disclosure;

FIG. 48 is a third side view of the implant of FIG. 44, in accordance with an aspect of the present disclosure;

FIG. 49 is a fourth side view of the implant of FIG. 44, in accordance with an aspect of the present disclosure;

FIG. 50 is a first end view of the implant of FIG. 44, in accordance with an aspect of the present disclosure;

FIG. 51 is a second end view of the implant of FIG. 44, in accordance with an aspect of the present disclosure;

FIG. 52 is a first cross-sectional view of the implant of FIG. 44 taken along line 52-52 in FIG. 50, in accordance with an aspect of the present disclosure;

FIG. 53 is a second cross-sectional view of the implant of FIG. 44 taken along line 53-53 in FIG. 50, in accordance with an aspect of the present disclosure;

FIG. 54 is a first perspective view of the anchor member of the orthopaedic compression anchor system of FIG. 34, in accordance with an aspect of the present disclosure;

FIG. 55 is a second perspective view of the anchor member of FIG. 54, in accordance with an aspect of the present disclosure;

FIG. 56 is a first side view of the anchor member of FIG. 54, in accordance with an aspect of the present disclosure;

FIG. 57 is a second side view of the anchor member of FIG. 54, in accordance with an aspect of the present disclosure;

FIG. 58 is a third side view of the anchor member of FIG. 54, in accordance with an aspect of the present disclosure;

FIG. 59 is a fourth side view of the anchor member of FIG. 54, in accordance with an aspect of the present disclosure;

FIG. 60 is a first end view of the anchor member of FIG. 54, in accordance with an aspect of the present disclosure;

FIG. 61 is a second end view of the anchor member of FIG. 54, in accordance with an aspect of the present disclosure;

FIG. 62 is a first cross-sectional view of the anchor member of FIG. 54 taken along line 62-62 in FIG. 60, in accordance with an aspect of the present disclosure;

FIG. 63 is a second cross-sectional view of the anchor member of FIG. 54 taken along line 63-63 in FIG. 60, in accordance with an aspect of the present disclosure;

FIG. 64 is a perspective view of the inserter of the orthopaedic compression anchor system of FIG. 34, in accordance with an aspect of the present disclosure;

FIG. 65 is a magnified view of a portion of the inserter of FIG. 64, in accordance with an aspect of the present disclosure;

FIG. 66 is a method of inserting an orthopaedic compression anchor of FIG. 34 into a patient's bone(s) and securing the suture anchor to a patient's soft tissue, in accordance with an aspect of the present disclosure;

FIG. 67 is a first perspective view of another inserter and another anchor member of an orthopaedic compression anchor system, in accordance with an aspect of the present disclosure;

FIG. 68 is a second perspective view of the inserter and anchor member of FIG. 67, in accordance with an aspect of the present disclosure;

FIG. 69 is a first perspective, magnified view of a portion of the inserter and the anchor member of FIG. 67, in accordance with an aspect of the present disclosure; and

FIG. 70 is a second perspective, magnified view of the portion of the inserter and the anchor member of FIG. 67, in accordance with an aspect of the present disclosure.

### DETAILED DESCRIPTION FOR CARRYING OUT THE INVENTION

Generally stated, disclosed herein are compression anchor systems, devices, instruments, and implants. Further, a method of assembly and a surgical method for using the compression anchor systems, devices, instruments, and implants are discussed.

In this detailed description and the following claims, the words proximal, distal, anterior, posterior, medial, lateral, superior and inferior are defined by their standard usage for indicating a particular part of a bone or implant according to the relative disposition of the natural bone or directional terms of reference. For example, “proximal” means the portion of a device or implant nearest the torso, while “distal” indicates the portion of the device or implant farthest from the torso. As for directional terms, “anterior” is a direction towards the front side of the body, “posterior” means a direction towards the back side of the body, “medial” means towards the midline of the body, “lateral” is a direction towards the sides or away from the midline of the body, “superior” means a direction above and “inferior” means a direction below another object or structure.

Similarly, positions or directions may be used herein with reference to anatomical structures or surfaces. For example, as the current devices and methods are described herein with reference to use with the bones of the hand, the bones of the hand, wrist and lower arm may be used to describe the surfaces, positions, directions or orientations of the devices, instrumentation and methods. Further, the devices and methods, and the aspects, components, features and the like thereof, disclosed herein are described with respect to one side of the body for brevity purposes. However, as the human body is relatively symmetrical or mirrored about a line of symmetry (midline), it is hereby expressly contemplated that the device and methods, and the aspects, components, features and the like thereof, described and/or illustrated herein may be changed, varied, modified, reconfigured or otherwise altered for use or association with another side of the body for a same or similar purpose without departing from the spirit and scope of the invention. For example, the devices and methods, and the aspects, components, features and the like thereof, described herein with respect to a right hand may be mirrored so that they likewise function with a left hand and vice versa. Further, the devices and methods, and the aspects, components, features and the like thereof, disclosed herein are described with respect to the hand for brevity purposes, but it should be understood that the devices and methods may be used with other bones of the body having similar structures, for example the lower extremity, and more specifically, with the bones of the ankle, foot, and leg.

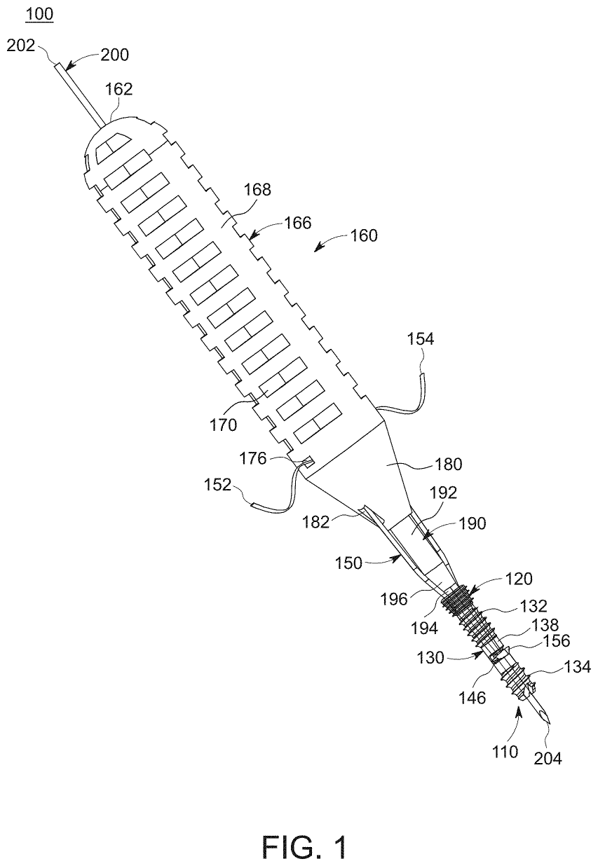

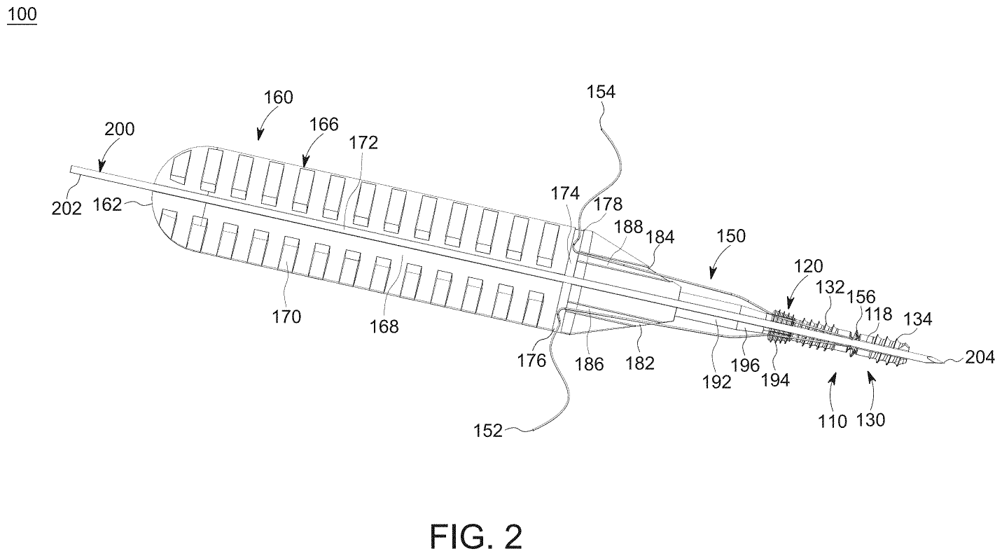

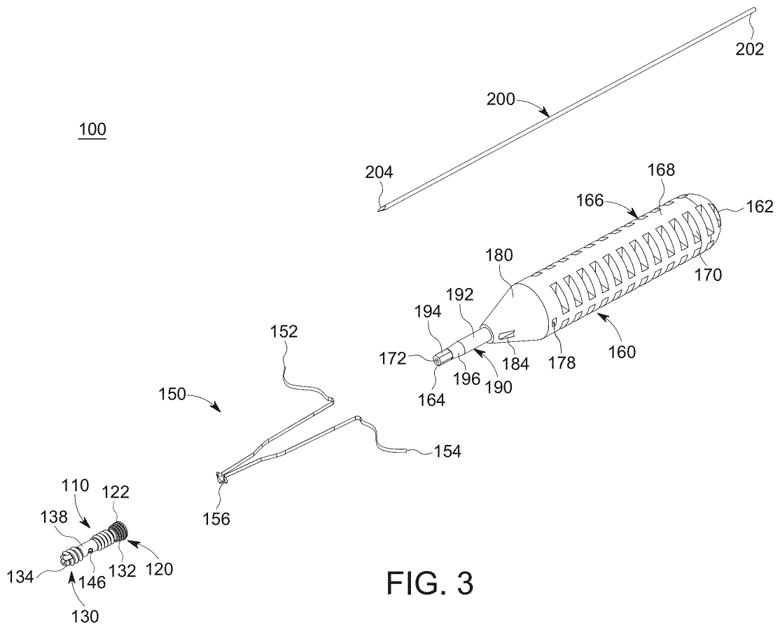

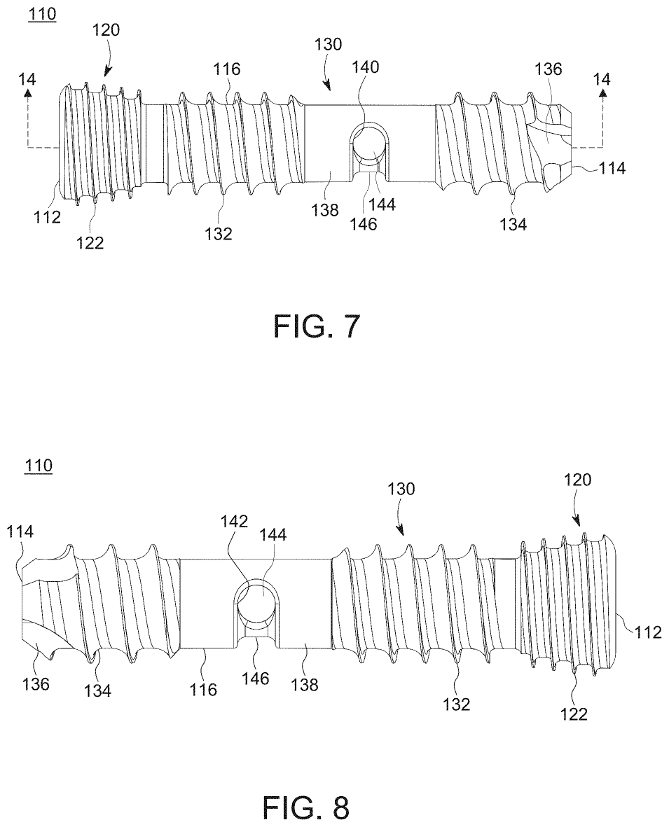

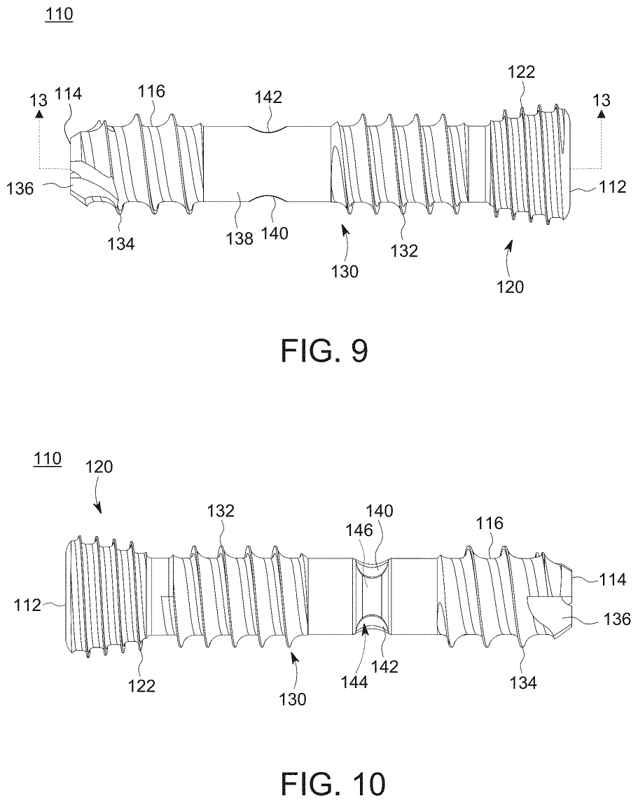

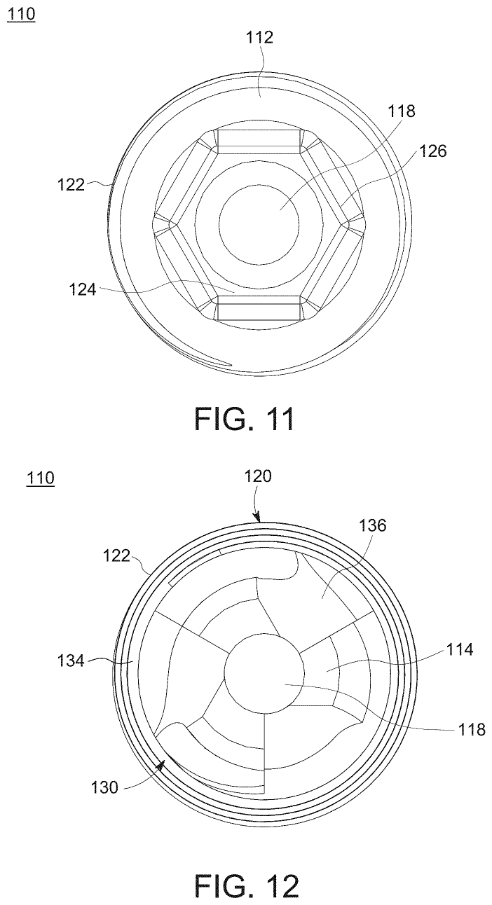

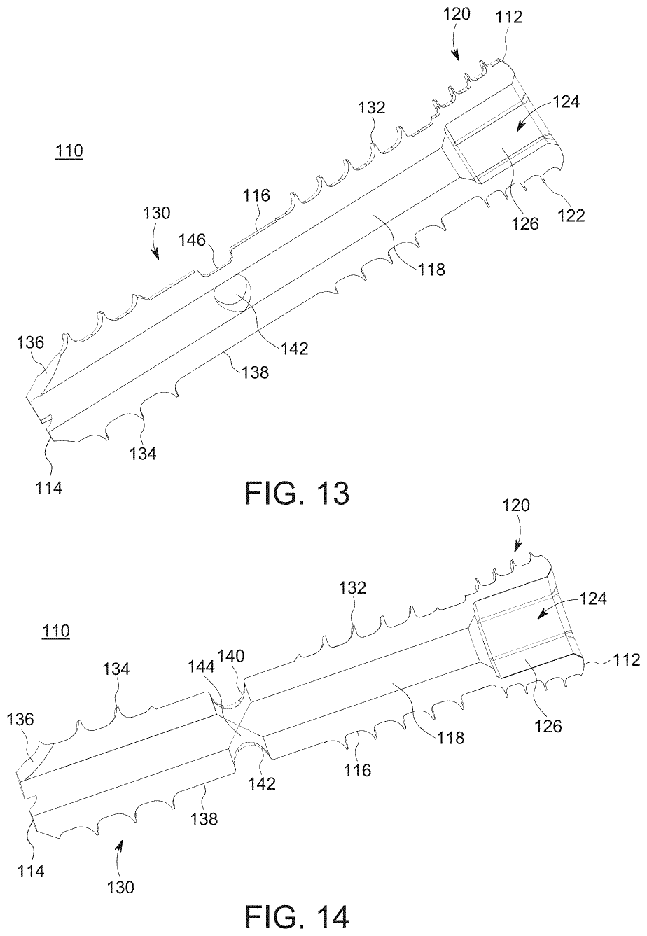

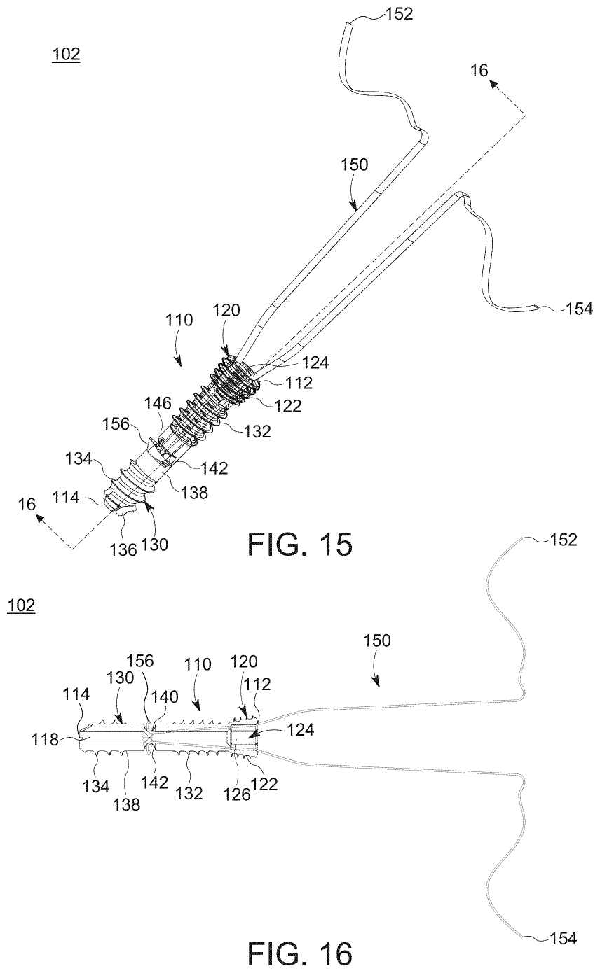

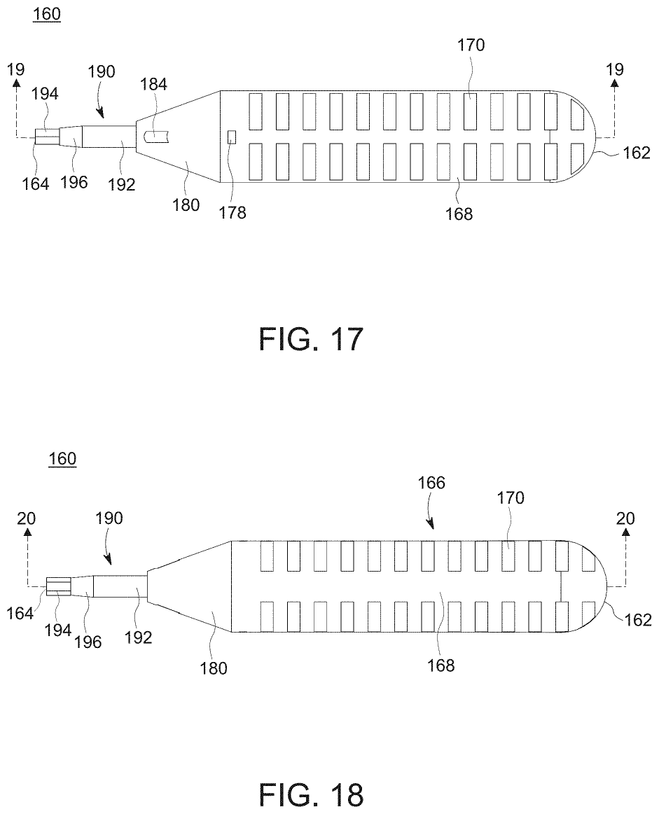

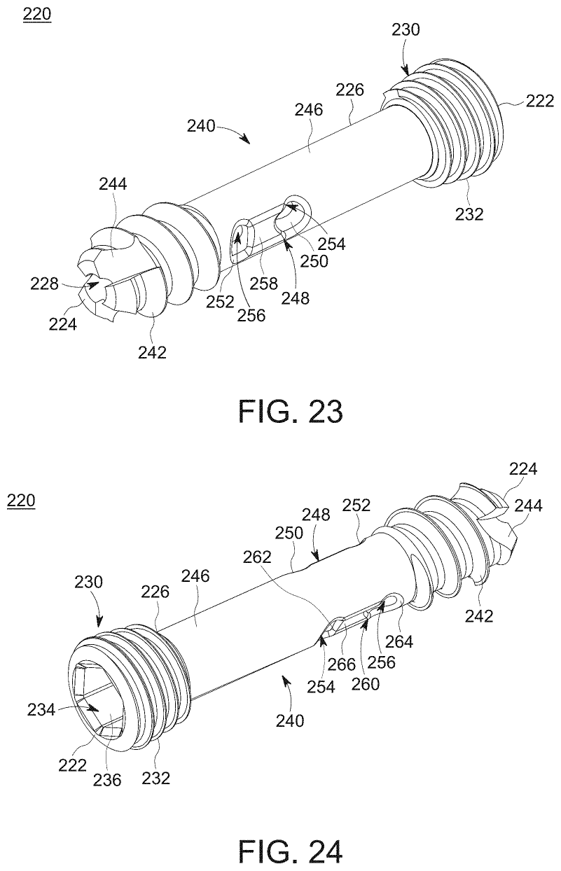

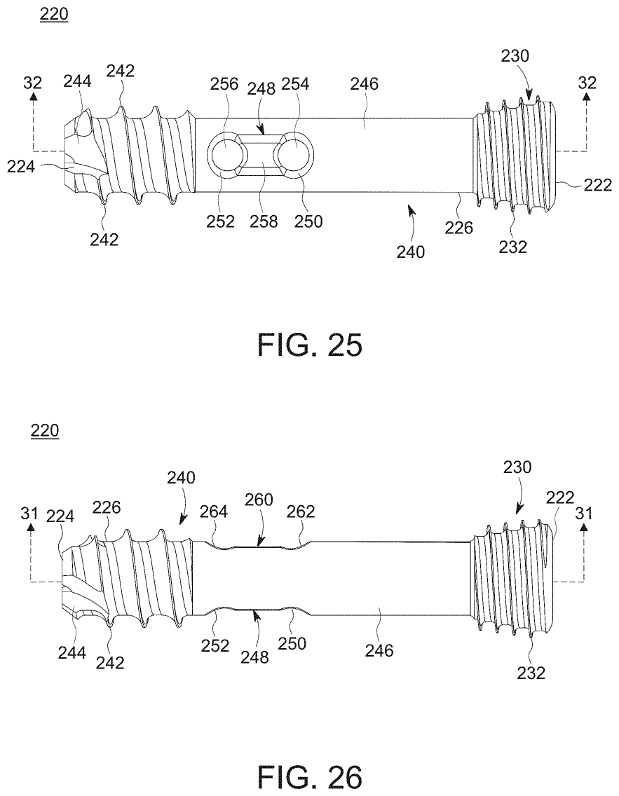

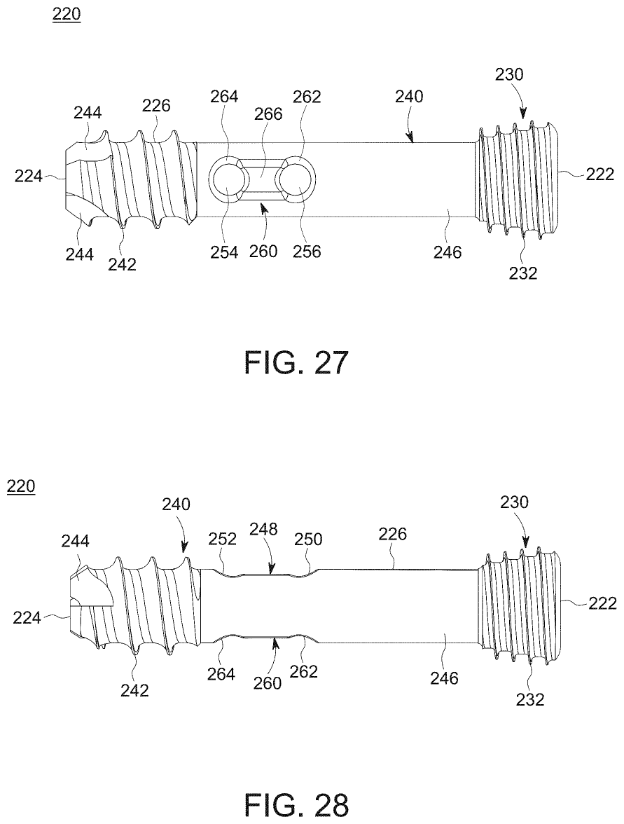

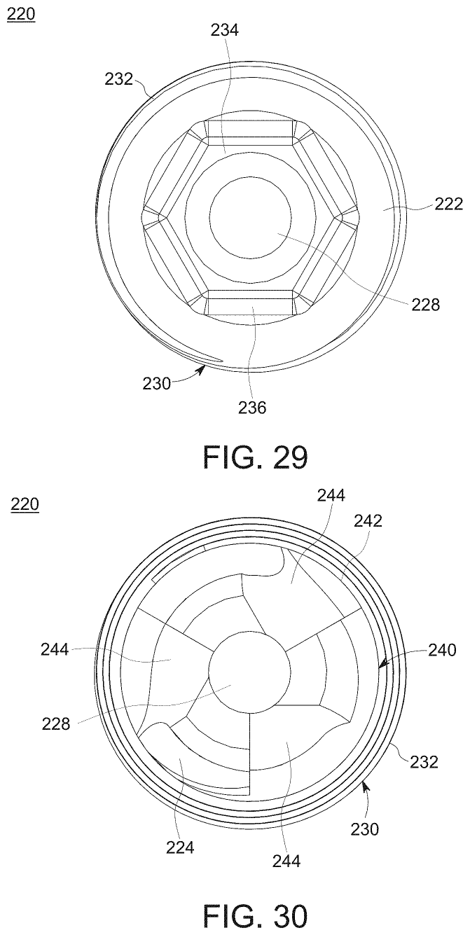

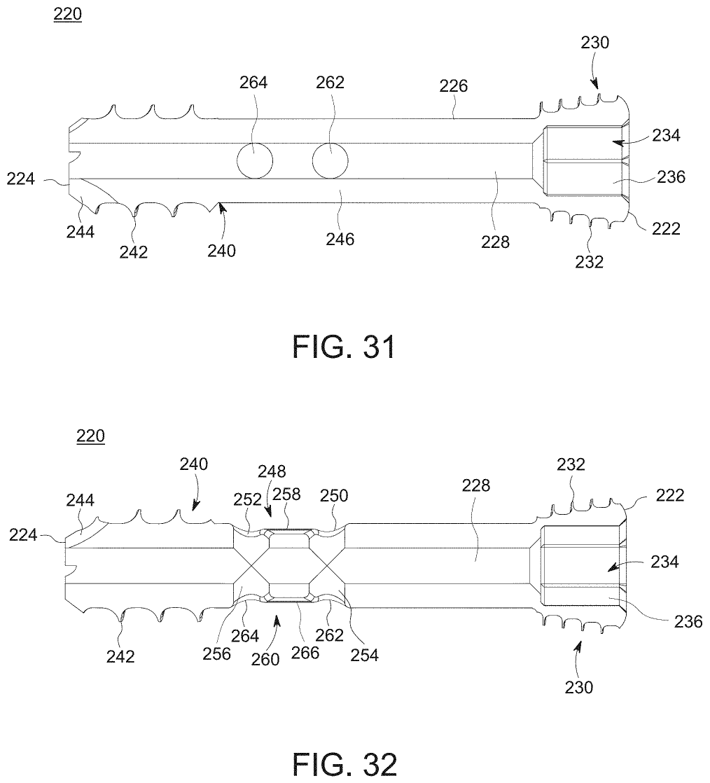

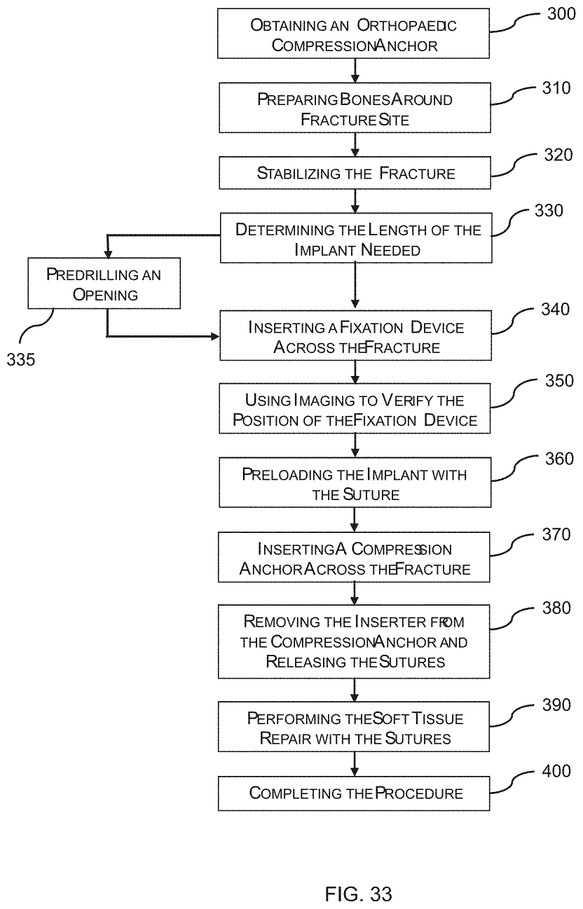

Referring to the drawings, wherein like reference numerals are used to indicate like or analogous components throughout the several views, and with particular reference to FIGS. 1-32, there are illustrated the devices of the orthopaedic compression anchor systems 100. In addition, the method of using the orthopaedic compression anchor systems 100 are shown in FIG. 33. One embodiment of an orthopaedic compression anchor system 100 may include, for example, an orthopaedic compression anchor 102, 210, an inserter 160, and k-wire or fixation devices 200. The compression anchor 102, 210 may include an implant 110, 220 and a suture anchor 150, 270.

In one embodiment, as shown in FIGS. 1-4, 15 and 16, a first compression anchor 102 is shown and includes implant 110 and a suture anchor 150. As shown in FIGS. 1-16, the first implant, bone screw, or compression screw 110 includes a first end 112 and a second end 114 opposite the first end 112. The implant 110 also includes an exterior surface 116 positioned on the outside of the implant 110. The exterior surface 116 extends circumferentially around the implant 110. In addition, the implant 110 also includes a cannulation or through hole 118 extending through the interior of the implant 110 from a first end 112 to a second end 114. The cannulation 118 may be, for example, sized and shaped to receive a k-wire or guide wire 200, as well as the suture strands of the suture anchor 150. The implant 110 also includes a head 120 at the first end 112 and a shaft 130 extending from the head 120 to the second end 114. The implant 110 may be, for example, a singular integral or monolithic piece (i.e., of one-piece construction), or may be formed from a plurality of components that are coupled (e.g., rigidly coupled) together to form the implant 110.

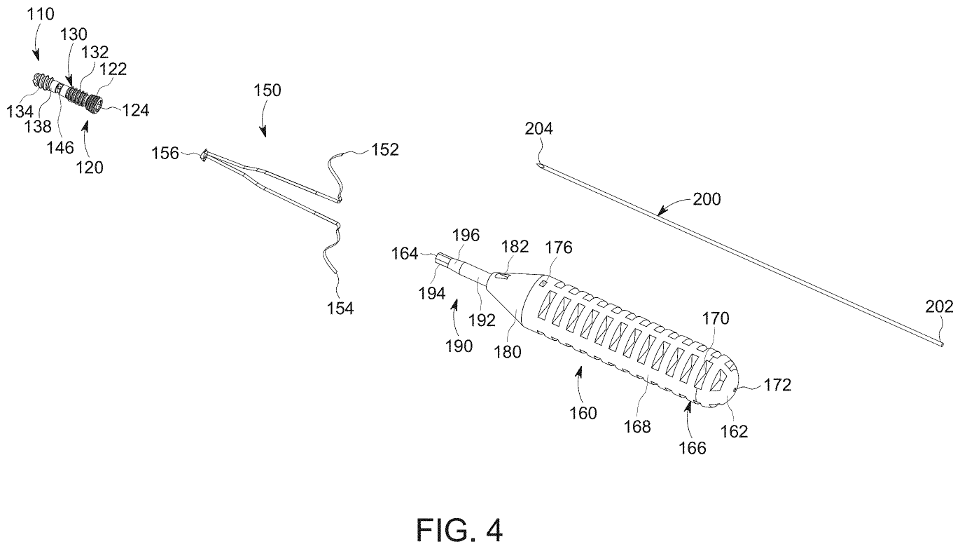

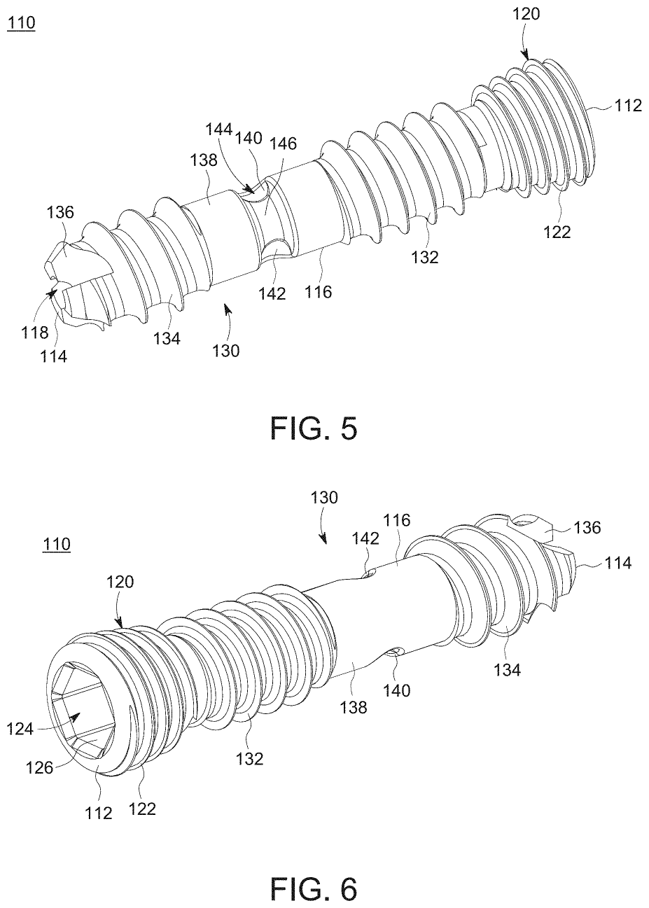

With continued reference to FIGS. 5-16, the head 120 may include a first exterior thread, threaded section, or trailing thread 122. The first exterior thread 122 may be, for example, a cortical thread. The first thread 122 may extend from, for example, the first end 112 of the implant 110 around the entire exterior surface of the head 120. It is also contemplated in alternative embodiments that the first thread 122 may extend, for example, around the circumference of the head 120 for only a portion between the first end 112 of the implant 110 to a second end of the head 120, where the head 120 couples to the shaft 130, and not the entirety of the exterior surface of the head 120. In still other embodiments, the first thread 122 may be, for example, more than one first thread 122 spaced apart from each other and positioned on the exterior surface of the head 120. As shown in FIGS. 4, 6, 11 and 13-16, the head 120 also includes a drive opening 124 extending into the head 120 from the first end 112. The drive opening 124 may, for example, overlap with the cannulation 118. The drive opening 124 may be formed by an interior drive surface 126. The interior drive surface 126 may have, for example, a polygonal shape, such as, a hexagonal shape, a torx shape, or another drive shape as known by one of ordinary skill in the art. The drive opening 124 and interior drive surface 126 may be configured or sized and shaped to receive a drive tool, such as, inserter 160. The head 120 may have, for example, a cylindrical or truncated cone shape.

Referring now to FIGS. 1-10 and 12-15, the shaft 130 includes at least one threaded section 132, 134 positioned along the length of the shaft 130. The at least one threaded section 132, 134 may be, for example, at least two threaded sections 132, 134, such as, at least one second threaded section, trailing thread, or trailing thread 132 and at least one third threaded section, leading thread, or leading trabecular thread 134. The trailing thread 132 and leading thread 124 may each be, for example, a trabecular threads. The second end 114 of the implant 110 may include flutes or cutouts 136 inset into the third threaded section 134. The shaft 130 may also include a smooth section 138 along the length of the shaft 130. The smooth section 138 may be positioned, for example, between the second threaded section 132 and the third threaded section 134. The lengths of the second threaded section 132, third threaded section 134, and smooth section 138 may be, for example, all the same, all different, or at least two the same and one different. In addition, the second threaded section 132 and the third threaded section 134 may have, for example, the same or different thread pitches. Further, the second threaded section 132 and third threaded section 134 may have, for example, the same or different thread pitches as the first threaded section 122.

The shaft 130 may also include a first opening 140 extending into the shaft 130. In addition, the shaft 130 may include a second opening 142 extending into the shaft 130 and positioned on a side opposite the first opening 140. The first and second openings 140, 142 may include, for example, a curved, chamfered, or filleted edge on the exterior surface 116. The first opening 140 may be connected to the second opening 142 by a through hole or suture eyelet 144. The through hole 144 may extend through the shaft 130 perpendicular to the cannulation 118. The first opening 140, second opening 142, and through hole 144 may be positioned along the length of the smooth section 138, as shown in FIGS. 5-10 and 14. In some embodiments, it is contemplated that the first opening 140 may be offset from the second opening 142 along the longitudinal axis or length of the shaft 130, such that the through hole 144 is positioned at an acute angle with respect to the cannulation and/or longitudinal axis. The shaft 130 may also include a groove or recess 146 inset into the exterior surface 116 of the shaft 130 and extending between the first opening 140 and the second opening 142 on at least one side of the shaft 130. The groove 146 may also be, for example, positioned on the exterior surface of the smooth section 138. The groove 146 may be, for example, aligned with the openings 140, 142 and the through hole 144 along the length of the implant 110. The first opening 140, second opening 142, through hole 144, and groove 146 may be, for example, positioned anywhere along the length of the shaft 130 between the head 120 and the second end 114 of the implant 110.

The suture anchor, guide thread, or surgical suture 150 are shown in FIGS. 1-4, 15 and 16. The suture anchor 150 may include a first end 152, a second end 154, and the anchored portion 156 positioned between the first end 152 and the second end 154. As shown in FIGS. 1, 2, 15 and 16, the suture anchor 150 may be inserted into the implant 110 such that the anchored portion 156 is positioned within the recess 146 with a first portion extending through the first opening 140, a portion of the suture anchor 150 extending between the anchored portion 156 and the first end 152 being positioned in the cannulation 118, and the first end 152 positioned outside of the implant 110. In addition, the anchored portion 156 may have a second portion extending through the second opening 142, a portion of the suture anchor 150 extending between the anchored portion 156 and the second end 154 being positioned in the cannulation 118, and the second end 154 positioned outside of the implant 110. The anchored portion 156 may be positioned, for example, anywhere along the length of the suture anchor 150 between the first end 152 and the second end 154. The suture anchor 150 may be, for example, preloaded into the implant 110 before the surgical procedure or loaded into the implant 110 during the surgical procedure. The suture anchor 150 may be preloaded by, for example, inserting the first end 152 of the suture anchor 150 into the first opening 140 and threading the first end 152 up the cannulation 118, through the drive opening 124, and out of the implant 110 and inserting the second end 154 of the suture anchor 150 into the second opening 142 and threading the second end 154 up the cannulation 118, through the drive opening 124, and out of the implant 110. Alternatively, one of the first end 152 or the second end 154 of the suture anchor 150 may be inserted through the drive opening 124, down the cannulation 118, and out one of the first and second openings 140, 142. Next, the end 152, 154 of the suture anchor 150 may be inserted into the other of the first and second openings 140, 142 and threaded up through the cannulation 118 and out through the drive opening 124.

Referring now to FIGS. 1-4 and 17-20, the inserter or driver 160 may include a first end 162 and a second end 164. The inserter 160 may include a handle 166 and a drive portion 190 coupled to and extending away from the handle 166. The handle 166 may extend from the first end 162 toward the second end 164 and the drive portion or drive feature 190 may extend from the second end 164 toward the first end 162. The handle 166 may have, for example, a cylindrical shape with a first diameter. The drive portion 190 may have, for example, a cylindrical shape along at least a portion of the length and at least one second diameter. The first diameter of the handle 166 may be, for example, larger than the at least one second diameter of the drive portion 190.

With continued reference to FIGS. 1-4 and 17-20, the handle 166 may include an exterior surface 168 on the outside of the handle 166. The exterior surface 168 may include, for example, a textured surface, which may include a plurality of recesses 170 or alternative textures to assist with gripping by a user. The inserter 160 may also include a cannulation 172 extending through the inserter 160 from the first end 162 to the second end 164. The cannulation 172 may extend from the first end 162, through the handle 166 and the drive portion 190 to the second end 164. The cannulation 172 extending through the inserter 160 may be, for example, sized and shaped or configured to receive a k-wire or guide wire 200. The handle 166 may also include a through hole 174 extending through the handle 166 perpendicular to a longitudinal axis of the handle 166 and the cannulation 172. The through hole 174 may be positioned near a second end of the handle 166. The through hole 174 may be accessed by a first exit opening 176 on a first side of the handle 166 and a second exit opening 178 on a second side of the handle 166. The first exit opening 176 may be, for example, positioned on the opposite side of the second exit opening 178. Thus, the through hole 174 connects the first exist opening 176 and the second exit opening 178.

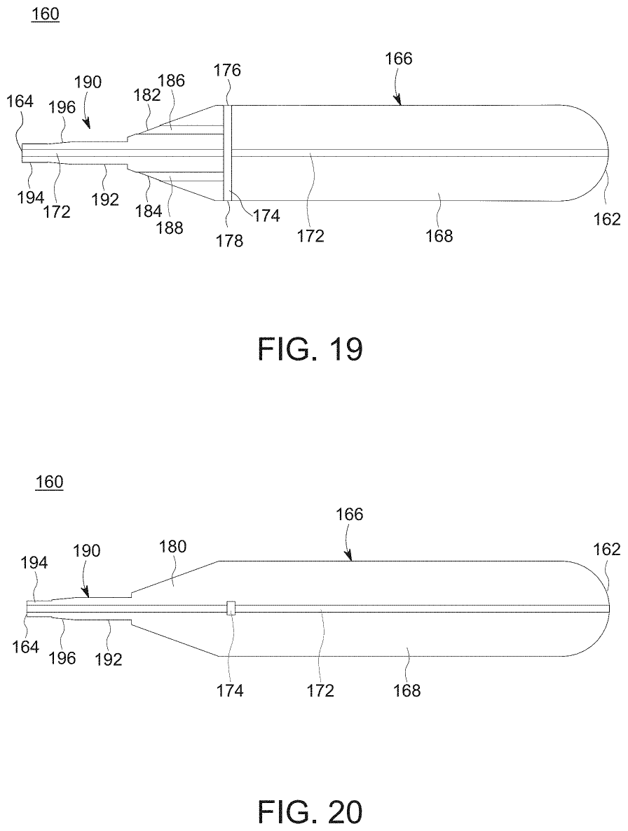

The handle 166 may also include a tapered region 180 extending from the second end of the handle 166 toward the second end 164 of the inserter 160, as shown in FIGS. 1-4 and 17-20. The tapered region 180 may couple the handle 166 to the drive portion 190. The tapered region 180 may also include a first entrance opening 182 extending into the tapered region 180 on a first side and a second entrance opening 184 extending into the tapered region 180 on a second side. The first entrance opening 182 being positioned on a side opposite the second entrance opening 184. The first entrance opening 182 may extend into a first channel 186, as shown in FIG. 19. The first channel 186 may extend, for example, parallel to the cannulation 172. The first channel 186 may also extend to and into communication with the through hole 174. The second entrance opening 184 may extend into a second channel 188. The second channel 188 may extend, for example, parallel to the cannulation 172, as shown in FIG. 19. The second channel 188 may also extend to and into communication with the through hole 174. The first and second channels 186, 188 both may be, for example, sized and shaped or configured to receive the suture strands of the suture anchor 150. The first and second channels 186, 188 may extend into the handle 166 from the second end of the handle 166 toward the first end 162.

As shown in FIGS. 1-4 and 17-20, the drive portion 190 may include a body 192 coupled to and extending from the tapered region 180 of the handle 166. The drive portion 190 may also include a drive member 194 positioned at the second end 164 of the inserter 160. The drive member 194 may be coupled to the body 192 by an intermediate portion 196. The intermediate portion 196 may be coupled to the body 192 on a first end and the drive member 194 on the second end. The intermediate portion 196 may be, for example, tapered from the body 192 to the drive member 194. The drive member 194 may have, for example, a polygonal shape, such as, a hexagonal shape, a torx shape, or another drive shape as known by one of ordinary skill in the art. The drive member 194 may be shaped to correspond to the drive opening 124 in the implant 110.

The guide wire or k-wire 200 is shown in FIGS. 1-4. The guide wire 200 may include a first end 202 and a second end 204. The second end 204 may be, for example, sharp or pointed to be inserted into at least one bone. In addition, the guide wire 200 may be sized and shaped to pass through the cannulation 172 of the inserter 160 and the cannulation 118 of the implant 110.

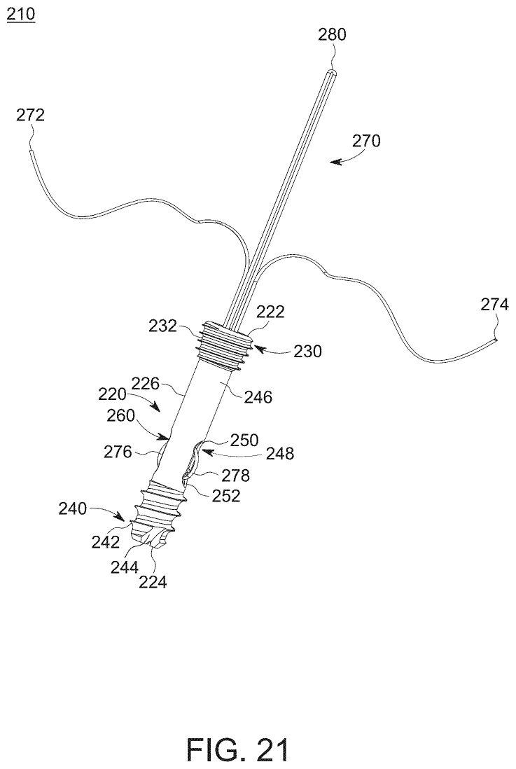

Referring now to FIGS. 21-32, another orthopaedic compression anchor 210 is shown. The compression anchor 210 may include an implant, bone screw, compression screw 220 and a suture anchor 270. The suture anchor 270 may be inserted into at least a portion of the implant 220 to secure the suture anchor 270 to the implant 220. The compression anchor 210 may be inserted using the inserter 160 and the guide wire 200 as described in greater detail below.

As shown in FIGS. 21-32, the implant 220 includes a first end 222 and a second end 224 opposite the first end 222. The implant 220 may also include an exterior surface 226 extending around the circumference of the implant 220 between the first end 222 and the second end 224. The implant 220 may have, for example, a generally cylindrical shape. In addition, the implant 220 may include a cannulation or through hole 228 extending through the implant 220 from the first end 222 to the second end 224. The implant 220 may include a head 230 extending from the first end 222 toward the second end 224 and a shaft 240 extending from the second end 224 toward the first end 222. The head 230 may be coupled to the shaft 240 between the first end 222 and the second end 224. The implant 220 may be, for example, a singular integral or monolithic piece (i.e., of one-piece construction), or may be formed from a plurality of components that are coupled (e.g., rigidly coupled) together to form the implant 220.

The head 230 may include a first exterior thread, threaded section, or trailing thread 232. The first thread 232 may be, for example, a cortical thread. The first thread 232 may extend from, for example, the first end 222 of the implant 220 around the entire exterior surface of the head 230. It is also contemplated in alternative embodiments that the first thread 232 may extend, for example, around the circumference of the head 230 for only a portion between the first end 222 of the implant 110 and a second end of the head 230, where the head 230 couples to the shaft 240. As shown in FIGS. 24 and 29-32, the head 230 may also include a drive opening 234 extending into the head 230 from the first end 222. The drive opening 234 may, for example, overlap with a portion of the cannulation 228 at the first end 112 of the implant 110. The drive opening 234 may be formed by an interior drive surface 236. The interior drive surface 236 may have, for example, a polygonal shape, such as, a hexagonal shape, a torx shape, or another drive shape as known by one of ordinary skill in the art. The drive opening 234 and interior drive surface 236 may be configured or sized and shaped to receive a drive tool, such as, inserter 160. In addition, the head 230 may have, for example, a cylindrical or truncated cone shape.

Referring now to FIGS. 21-28, 31 and 32, the shaft 240 may include at least one second exterior thread, threaded section, leading thread 242. The at least one second threaded section 242 may be, for example, positioned at the second end 224 of the implant 220. The at least one second threaded section 242 may be, for example, a cortical thread. The second end 224 of the implant 220 may also include flutes or cutouts 244. The flutes 244 may be, for example, inset into the second threaded section 242. In addition, the shaft 240 may include a smooth section 246 along the length of the shaft 240. The smooth section 246 may be positioned, for example, between the first threaded section 232 on the head 230 and the second threaded section 242 on the shaft 230. The length of the second threaded section 242 and the smooth section 246 may be, for example, the same or different. As shown in the depicted embodiment, the smooth section 246 has a length larger than the length of the second threaded section 242 along the shaft 240 of the implant 220. In addition, the first threaded section 232 and the second threaded section 242 may have, for example, the same or different thread pitches.

The shaft 130 may also include a first anchor 248 on a first side of the shaft 240 and a second anchor 260 on a second side of the shaft 240. The first anchor 248 may be, for example, positioned opposite the second anchor 260. It is also contemplated that in alternative embodiments the first anchor 248 and the second anchor 260 may be offset from each other along the longitudinal axis of the implant 220 and/or offset from each other around the circumference of the implant 220, such that the first anchor 248 and the second anchor 260 are not directly opposite each other. The first anchor 248 may include a first opening 250 extending from the exterior surface 226 of the implant 220 into the cannulation 228. The first anchor 248 may also include a second opening 252 extending from the exterior surface 226 of the implant 220 into the cannulation 228. The first opening 250 may be spaced apart from the second opening 252 along the longitudinal axis of the implant 220. The first and second openings 250, 252 may have, for example, a curved, chamfered, or filleted edge on the exterior surface 226. The shaft 240 may also include a first through hole 254 extending through the shaft 240 from the first opening 250 to the opposite side of the shaft 240. The shaft 240 may further include a second through hole 256 extending through the shaft 240 from the second opening 252 to the opposite side of the shaft 240. The first and second through holes 254, 256 may extend through the implant 220, for example, perpendicular to the cannulation 228. The first anchor 248 may also include a first recess or groove 258 inset into the exterior surface 226 of the implant 220 and connecting the first opening 250 and the second opening 252. The first recess 258 may be positioned on the smooth section 246 of the implant 220. The first recess 258 may be, for example, aligned with the openings 250, 252 and extend parallel to the cannulation 228.

The second anchor 260 may include a third opening 262 and a fourth opening 264. The third and fourth openings 262, 264 may extend from the exterior surface 226 of the implant 220 into the cannulation 228. The third and fourth openings 262, 264 may extend through the implant 220, for example, perpendicular to the cannulation 228. The third opening 262 may be spaced apart from the fourth opening 264 along the longitudinal axis of the implant 220. The third and fourth openings 262, 264 may have, for example, a curved, chamfered, or filleted edge on the exterior surface 226. The third opening 262 may extend into the first through hole 254 such that the first through hole 254 connects the first opening 252 and the third opening 262. The fourth opening 264 may extend into the second through hole 256 such that the second through hole 256 connects the second opening 254 and the fourth opening 264. The second anchor 260 may also include a second recess or groove 266 inset into the exterior surface 226 of the implant 220 and connecting the third opening 262 and fourth opening 264. The second recess 266 may be positioned, for example, on the smooth section 246 of the implant 220. The second recess 266 may be, for example, aligned with the openings 262, 264 and extend parallel to the cannulation 228.

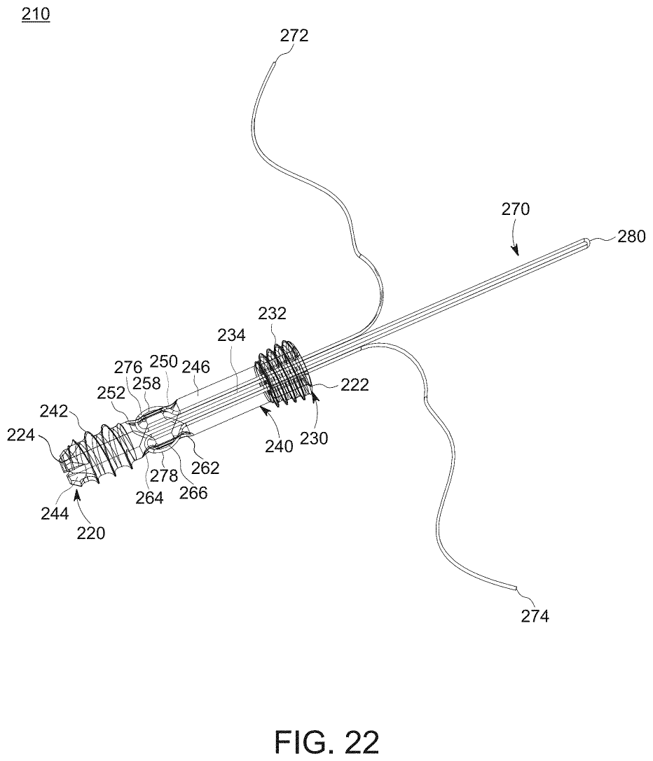

As shown in FIGS. 21 and 22, the suture anchor, guide thread, or surgical suture 270 includes a first end 272 and a second end 274 opposite the first end 272. The suture anchor 270 may also include a first anchor portion 276 positioned between the first end 272 and the second end 274 and a second anchor portion 278 positioned between the first anchor portion 276 and the second end 274. The suture anchor 270 may be inserted into the implant 220 such that the first anchor portion 276 is positioned within the first recess 258 and the second anchor portion 260 is positioned within the second recess 266. The first anchor portion 276 may extend from the first end 272, through the cannulation 228, out the first opening 250, back in the second opening 252, and out of the cannulation 228 to a tissue coupling portion 280. The second anchor portion 278 may extend from the second end 274, through the cannulation 228, out the third opening 262, back in the fourth opening 264, and out of the cannulation 228 to the tissue coupling portion 280. The tissue coupling portion 280 forms a loop between the first anchor portion 276 and the second anchor portion 278 that may be used, for example, to secure the soft tissue back to the hard tissue or bone (not shown).

The suture anchor 270 may be, for example, fully preloaded into the implant 220 before the surgical procedure, as shown in FIGS. 21 and 22, partially preloaded before the surgical procedure, or fully loaded into the implant 220 during the surgical procedure. The suture anchor 270 may be preloaded by, for example, inserting the first end of the suture anchor 270 through the first opening 250, into the cannulation 228 and out the first end 222 of the implant 220. Preloading may also include inserting the second end 274 of the suture anchor 270 through the cannulation 228, out the fourth opening 264, back in the third opening 262, through the cannulation 228 and out the first end 222 of the implant 220. The insertion of the suture anchor 270 may occur in any order that would position the first anchor portion 276 within the first anchor 248 and the second anchor portion 278 within the second anchor 260, as shown in FIGS. 21 and 22. For a partially preloaded implant 220, the suture anchor 270 may be inserted into one of the first anchor 248 and the second anchor 260 leaving one free end 272, 274 of the suture anchor 270 to be inserted into the other anchor 248, 260 during the surgical procedure.

Referring now to FIG. 33, a surgical method of implanting an orthopaedic compression anchor 102, 210 is shown. The surgical method may include obtaining an orthopaedic compression anchor 300. In addition, the method may include preparing the bones surrounding the fracture site as appropriate for the specific fracture or fusion 310. Once the bones are prepared, the fracture can be stabilized 320 and the length of the screw required should be measured 330. Next, an opening may optionally be predrilled into cortical bone 335. A fixation device, k-wire, guide wire, or the like may then be inserted across the fracture 340 and an imagining device, such as a mini C-arm, may be used to verify the position of the k-wire 350. In another embodiment, the k-wire could be inserted before predrilling the bone. The compression anchor may then be preloaded with the suture, if the compression screw will also be used as an anchor for ligamentous, tendon, or other soft tissue repair 360. The compression anchor may then be inserted across the fracture and through the predrilled opening, if made, to maintain compression across the fracture 370. The compression anchor may be inserted with a standard torx or hex driver or a custom inserter tool. The custom inserter tool will be configured to manage the suture. Thus, if using the inserter 160, the suture ends 152, 154, 272, 274 should be inserted through a channel 186, 188, into the through hole 174, and out of the handle 166 through the exit openings 176, 178, respectively. Once the compression anchor is in the desired position, the inserter may be pulled out and the sutures may be released 380. After the sutures are released, the sutures may be loaded into needles for performing the repair 390. The ligament, tendon and/or soft tissue repair may then be completed including closing any incisions made during the repair 400.

The surgical method may be used for articular fractures and other fractures to obtain compression across the fracture line while allowing for congruent cartilaginous surfaces to remain intact. The suture coupled to the implant allows for soft tissue repair in the area of the fracture without the need for a separate suture anchor. In one embodiment, the orthopaedic compression anchors 102, 210 may be used for repairing a scaphoid fracture and associated soft tissue injuries. In other embodiments, the orthopaedic compression anchors 102, 210 may be used for repairing other fractures of the bones of the hand, wrist, foot, ankle, and other like bones.

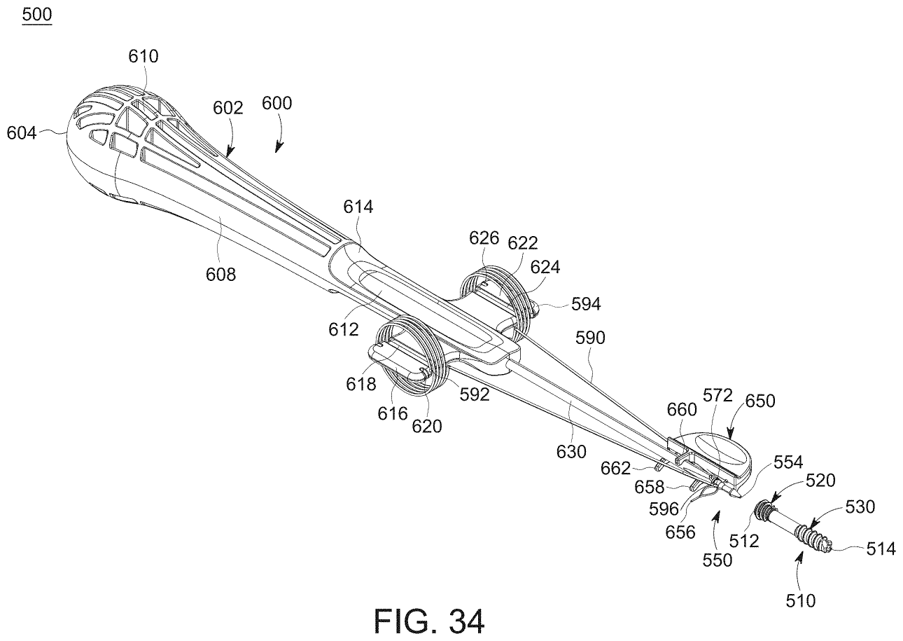

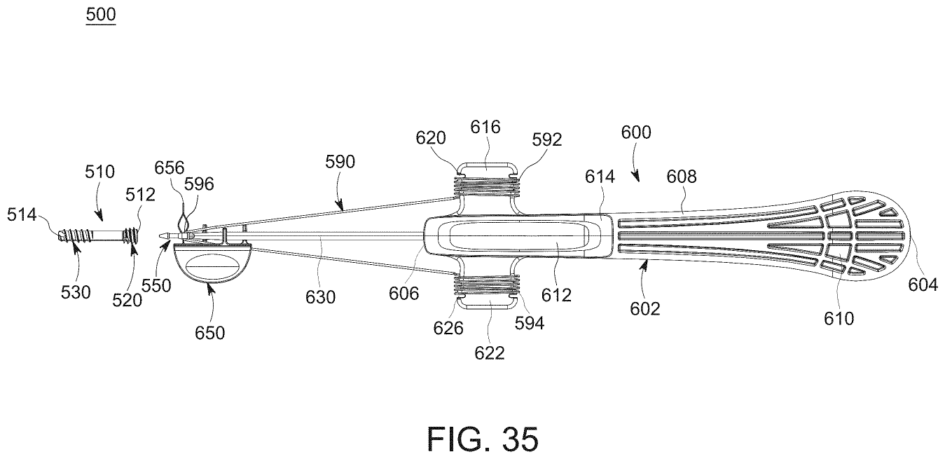

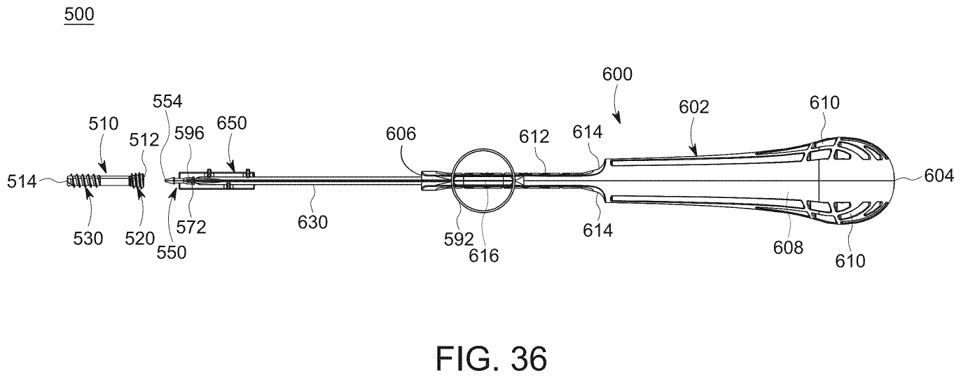

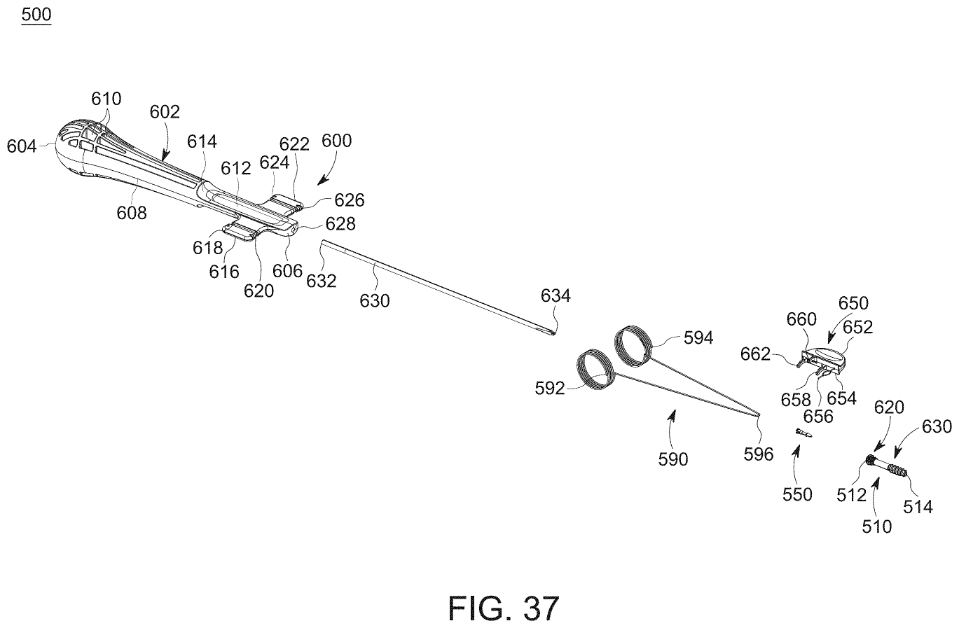

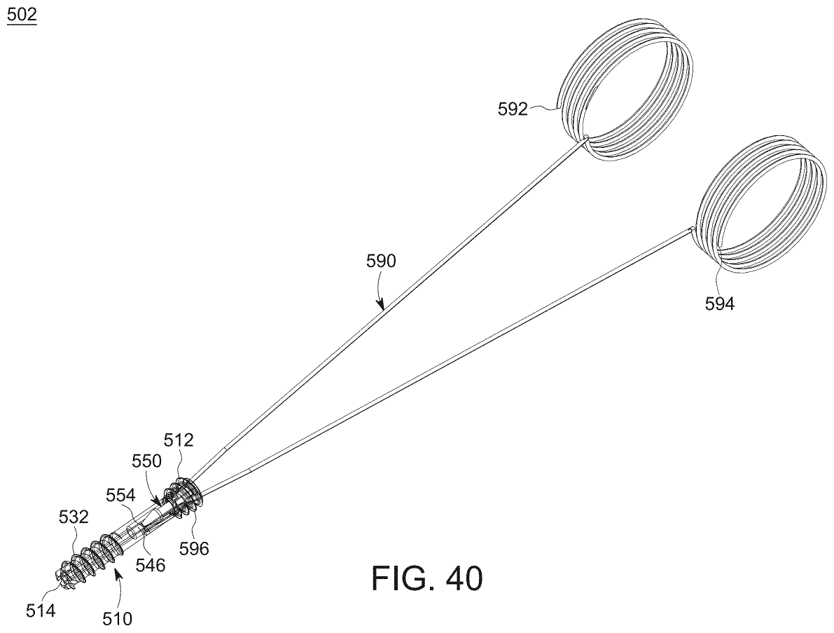

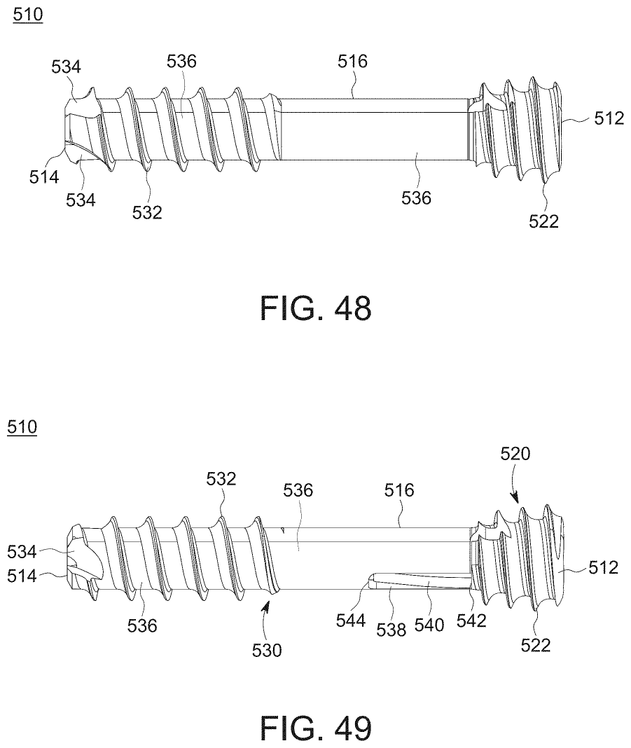

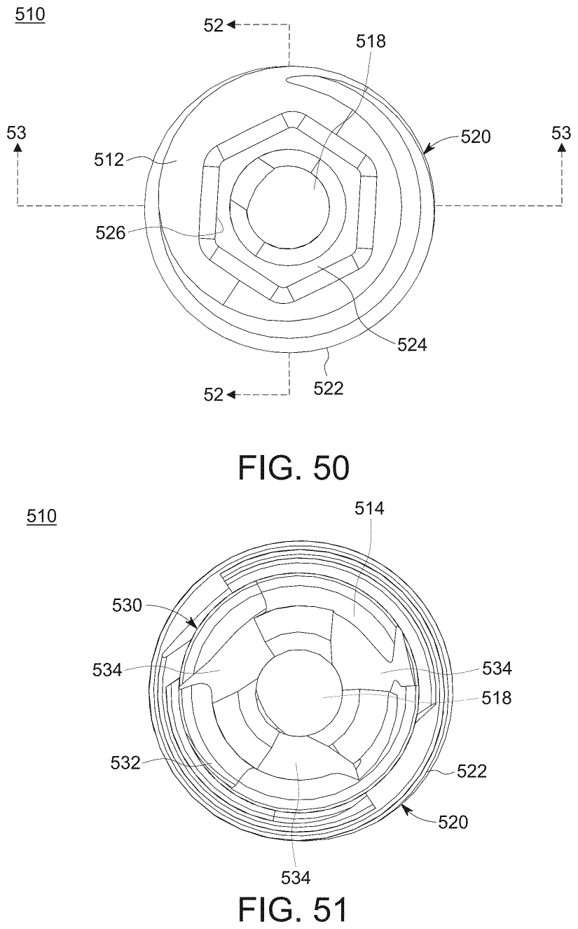

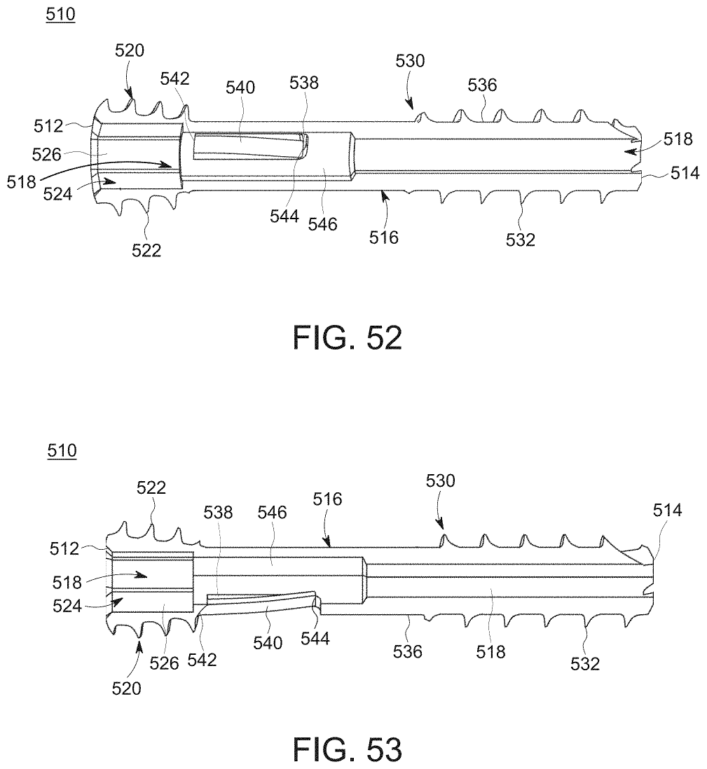

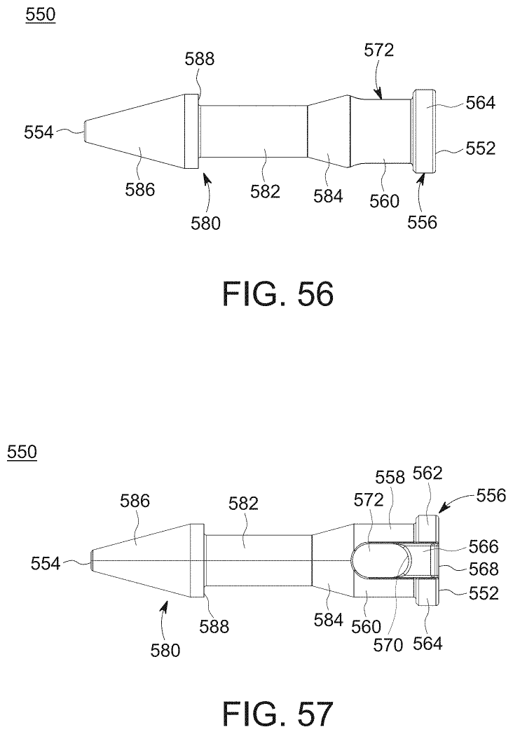

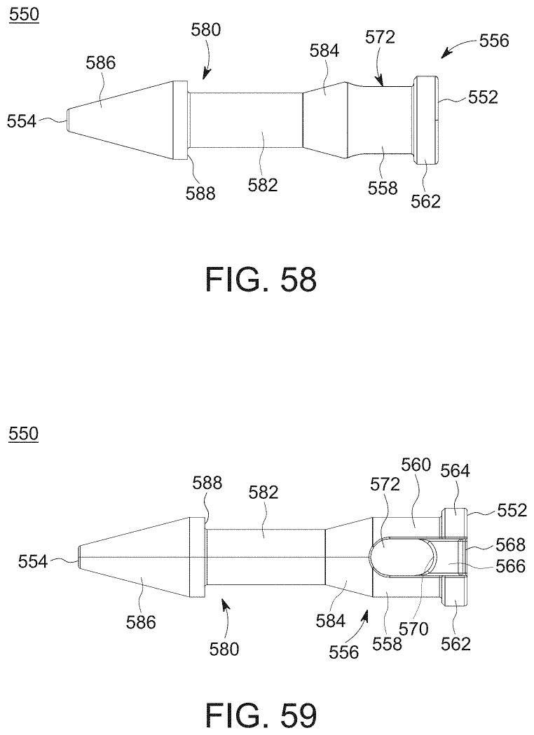

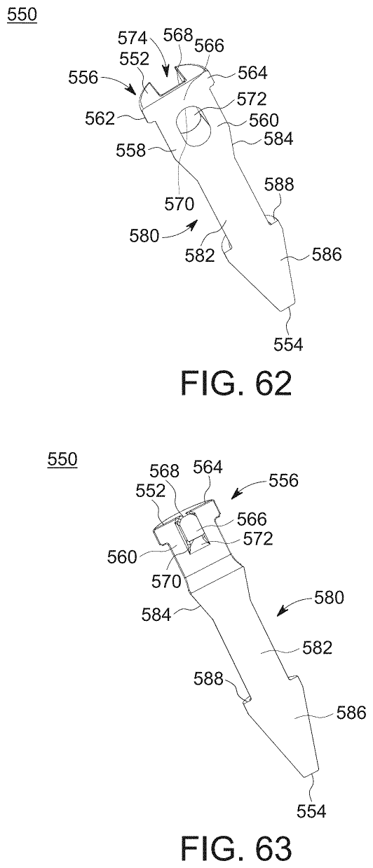

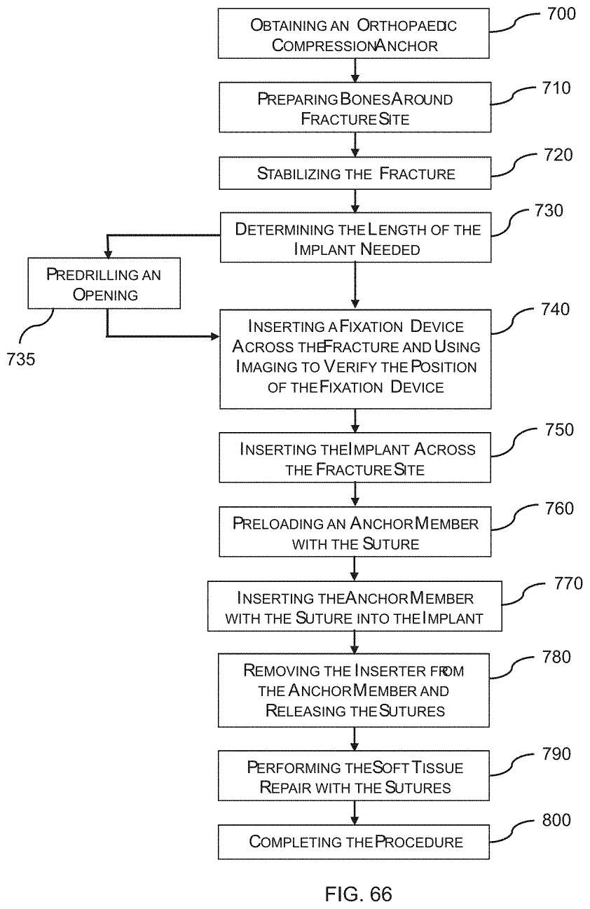

Referring to the drawings, wherein like reference numerals are used to indicate like or analogous components throughout the several views, and with particular reference to FIGS. 34-63, there are illustrated the devices of another orthopaedic compression anchor system 500. In addition, the method of using the orthopaedic compression anchor system 500 is shown in FIG. 66. One embodiment of an orthopaedic compression anchor system 500 may include, for example, an orthopaedic compression anchor 502, an inserter 600. The compression anchor 502 may include an implant 510, an anchor member 550, and a suture anchor 590. The compression anchor system 500 may also include a k-wire or other fixation device (not shown).

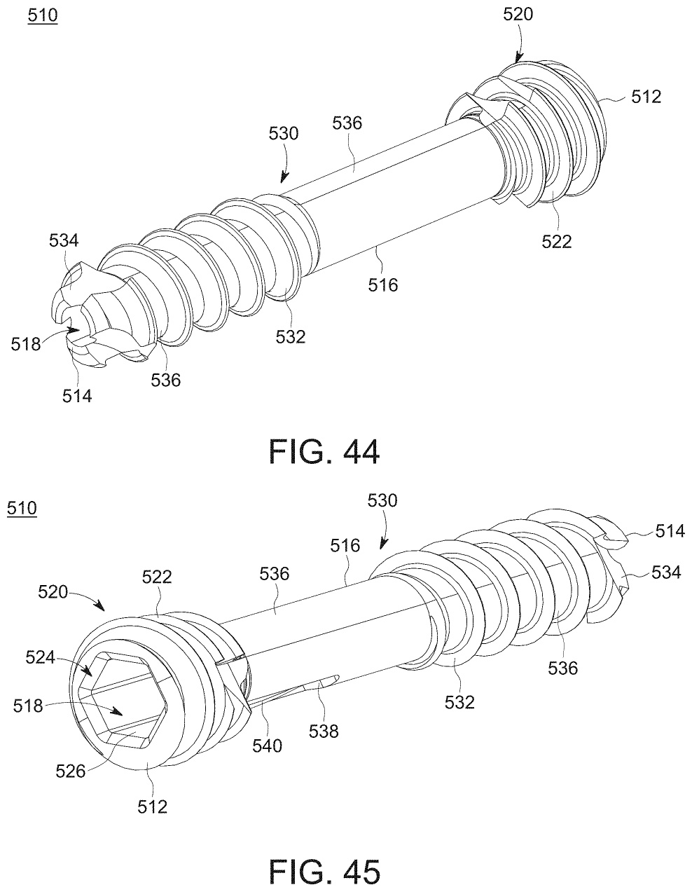

In one embodiment, as shown in FIGS. 34-43, the compression anchor 502 is shown and includes the implant 510, the anchor member 550, and a suture anchor 590. With continued reference to FIGS. 34-43 and reference to FIGS. 44-53, the third implant, bone screw, or compression screw 510 includes a first end 512 and a second end 514 opposite the first end 512. The implant 510 also includes an exterior surface 516 positioned on the outside of the implant 510. The exterior surface 516 extends circumferentially around the implant 510. In addition, the implant 510 also includes a cannulation or through hole 518 extending through the interior of the implant 510 from the first end 512 to the second end 514. The cannulation 518 may be, for example, sized and shaped to receive a k-wire, guide wire, or other fixation device (not shown), as well as a portion of the suture strands of the suture anchor 590. The implant 510 also includes a head 520 at the first end 512 and a shaft 530 extending from the head 520 to the second end 514. The implant 510 may be, for example, a singular integral or monolithic piece (i.e., of one-piece construction), or may be formed from a plurality of components that are coupled (e.g., rigidly coupled) together to form the implant 510.

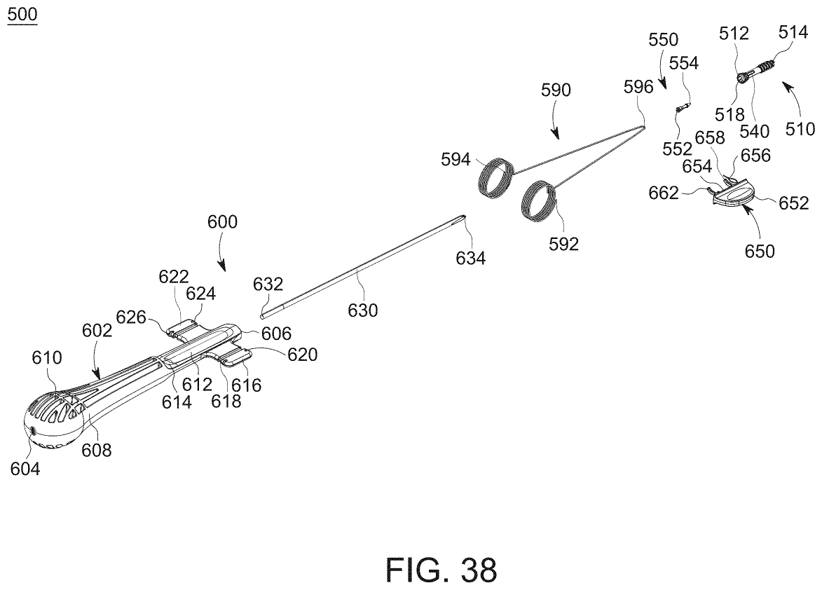

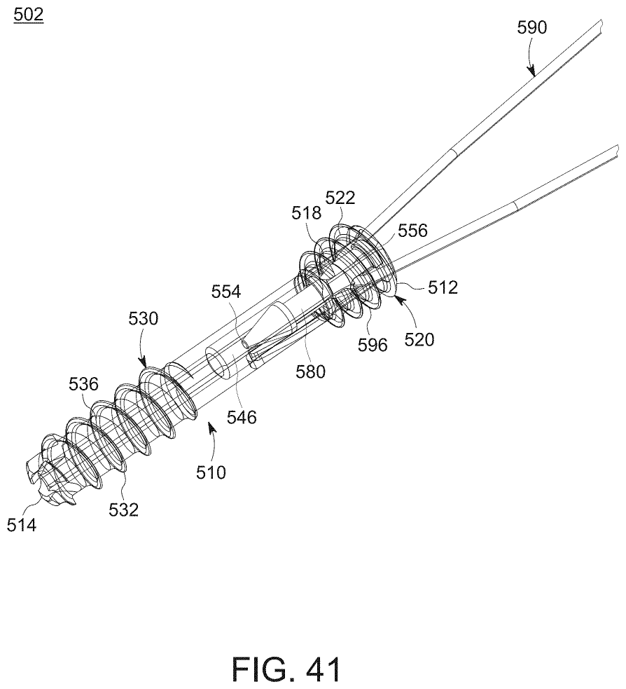

With continued reference to FIGS. 41-53, the head 520 may include a first exterior thread, threaded section, or trailing thread 522. The first exterior thread 522 may be, for example, a cortical thread. The first thread 522 may extend from, for example, the first end 512 of the implant 510 to the shaft 530 around the entire exterior surface of the head 520. It is also contemplated in alternative embodiments that the first thread 522 may extend, for example, around the circumference of the head 520 for only a portion between the first end 512 of the implant 510 to a second end of the head 520, where the head 520 couples to the shaft 530, and not around the entirety of the exterior surface of the head 520. In still other embodiments, the first thread 522 may be, for example, more than one first thread 522 spaced apart from each other and positioned on the exterior surface of the head 520. As shown in FIGS. 38, 42-43, 45, 50 and 52-53, the head 520 also includes a drive opening 524 extending into the head 520 from the first end 512. The drive opening 524 may, for example, overlap with the cannulation 518. The drive opening 524 may be formed by an interior drive surface 526. The interior drive surface 526 may have, for example, a polygonal shape, such as, a hexagonal shape, a torx shape, or another drive shape as known by one of ordinary skill in the art. The drive opening 524 and interior drive surface 526 may be configured or sized and shaped to receive a drive tool (not shown). The head 520 may have, for example, a cylindrical or truncated cone shape. The head 520 may taper from the first end 512 to the shaft 530.

Referring now to FIGS. 34-49 and 51-53, the shaft 530 includes at least one second threaded section 532 positioned along the length of the shaft 530. As shown, the at least one second threaded section 532 may be, for example, one threaded section or trabecular thread 532 extending from the second end 514 of the implant 510 toward the head 520 along the shaft 530. The second end 514 of the implant 510 may include flutes or cutouts 534 inset into the threaded section 532. The shaft 530 may also include a smooth section 536 along the length of the shaft 530. The smooth section 536 may be positioned, for example, between the threaded section 532 and the head 520. The lengths of the threaded section 532 and smooth section 536 may be, for example, the same or different. Further, the second threaded section 532 may have, for example, the same or different thread pitch as the first threaded section 522.

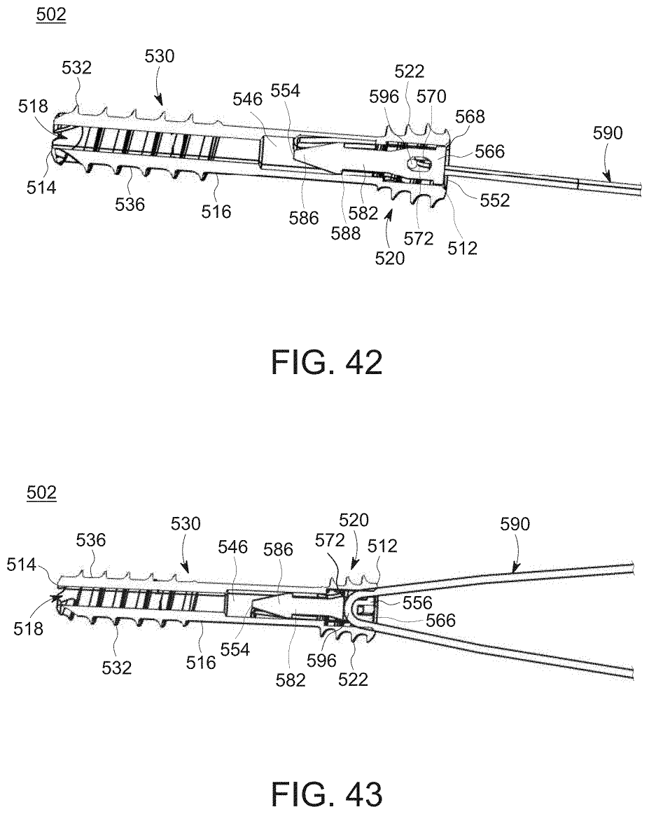

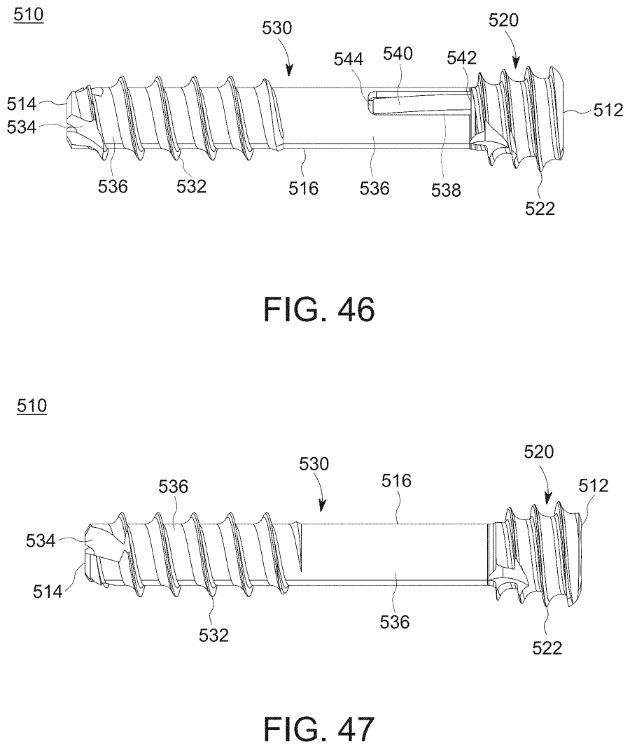

The shaft 530 may also include an opening 538 extending into the shaft 530 from an exterior surface 516 of the implant 510 and into the through hole 518. The opening 538 may have, for example, a rectangular or oval shape. The opening 538 may also have, for example, a length extending along the length of the implant 510 which extends from the first end 512 to the second end 514 that is longer than its width extending around the circumference of the implant 510. The opening 538 may include, for example, a curved, chamfered, or filleted edges on the exterior surface 516. The shaft 530 may also include an engagement member, tab, leaf spring, or locking member 540 coupled to the second end of the head 520 and extending into the opening 538 along the longitudinal axis or length extending from the first end 512 to the second end 514 of the implant 510. The engagement member 540 has a first end 542 and a second end 544 opposite the first end 542. The engagement member 540 may also include an interior surface 546 extending between the first end 542 and the second end 544 and positioned on the inside adjacent to the through hole 518. The first end 542 may be, for example, coupled to or integral with the second end of the head 520, as shown in FIGS. 42 and 46. The second end 544 may be, for example, a free end or movable end that allows for deformation upon the insertion of the anchor member 550 and engagement with the anchor member 550 once inserted into the implant 510. As shown, the second end 544 of the engagement member 540 is not coupled to any surrounding portion of the shaft 530. However, it is also contemplated that the second end 544 of the engagement member 540 could have a moveable or flexible connection (not shown) between the second end 544 of the engagement member 540 and a portion of the shaft 530 surround the opening 538. The moveable or flexible connection (not shown) would allow for the engagement member 540 to extend out of the opening 538 during insertion of the anchor member 550 and then for the second end 544 of the engagement member 540 to return to a position within the through hole 518 of the implant 510 to engage a portion of the anchor member 550 and retain the anchor member 550 within the implant 510. The engagement member 540 may have, for example, a rectangular or oval shape with a length extending between the first end 542 and the second end 544 that is longer than the width extending perpendicular to the longitudinal axis or length extending between the first end 542 and the second end 544.

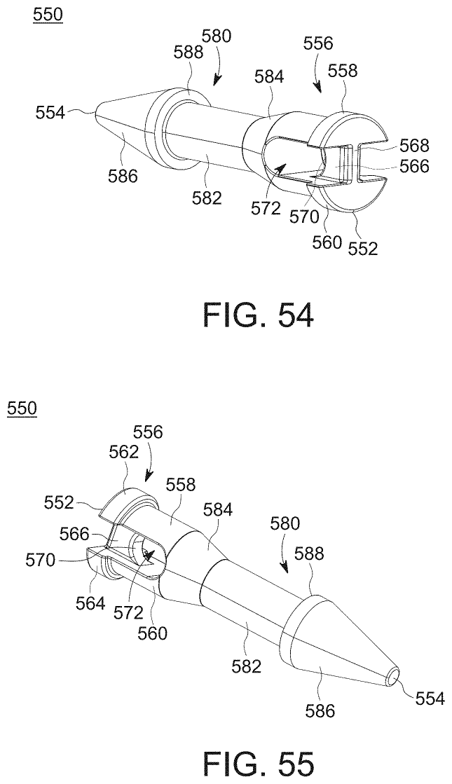

Referring now to FIGS. 34-43 and 54-63, the anchor member or plug 550 is shown. The anchor member 550 may include a first end 552 and a second end 554. A head 556 may be positioned at the first end 552 and a shaft 580 may extend from the head 556 to the second end 554 of the anchor member 550. The head 556 may have a first arm 558, a second arm 560, and a bridge 566 extending between and coupled to a portion of the first arm 558 and a portion of the second arm 560, as shown in FIGS. 54-59 and 61-63. The first arm 558 of the head 556 may also include a first projection 562 positioned at the first end 552 of the anchor member 550. The first projection 562 may extend from the exterior surface of the first arm 558 forming an engagement surface on the bottom side of the first projection 562 and a top surface at the first end 552 of the anchor member 550. The first projection 562 may, for example, extend around the entire exterior surface of the rest of the first arm 558. The first projection 562 may have, for example, a semi-circular or curved shape to correspond to the semi-circular or curved shape of the first arm 558. The second arm 560 of the head 556 may also include a second projection 564 positioned at the first end 552 of the anchor member 550. The second projection 564 may be positioned on an opposite side of the head 556 than the first projection 562 and the first projection 562 and second projection 564 may be, for example, mirror images of each other. The second projection 562 may extend from the exterior surface of the second arm 560 forming an engagement surface on the bottom side of the second projection 564 and a top surface at the first end 552 of the anchor member 550. The second projection 564 may, for example, extend around the entire exterior surface of the rest of the second arm 560. The second projection 564 may also have, for example, a semi-circular or curved shape to correspond to the semi-circular or curved shape of the second arm 560.

A portion of the first arm 558 and a portion of the second arm 560 may also be separated by, for example, a through hole 572 extending through the head 556, as shown in FIGS. 54-55, 57, 59, and 62-63. The through hole 572 may be, for example, positioned adjacent and distal to the bridge 566. The bridge 566 may include a top surface 568 and a bottom surface 570. The top surface 568 may be, for example, flat, planar or the like. The top surface 568 may extend between the top surfaces of the first and second projections 562, 564 to form the first end 552 of the anchor member 550. Each of the surfaces forming the first end 552 of the anchor member 550 may be flat or planar forming a uniform or flush surface. The bottom surface 570 of the bridge 566 may be, for example, curved or arced to form a semi-circular shape. The bottom surface 570 may also include chamfered, curved, or filleted edges to provide a smooth surface for receiving the suture 590. The shape of the bottom surface 570 of the bridge 566 may also, for example, match or correspond to the shape of the suture 590 to be received within the through hole 572 of the head 556. The bottom surface 570 of the bridge 566 forms a top side of the through hole 572. The bottom surface of the through hole 572 may also be, for example, curved or arced to form a semi-circular shape. The bottom surface of the through hole 572 may also be shaped to, for example, match or correspond to the shape of the suture 590 to be received within the through hole 572 of the head 556.

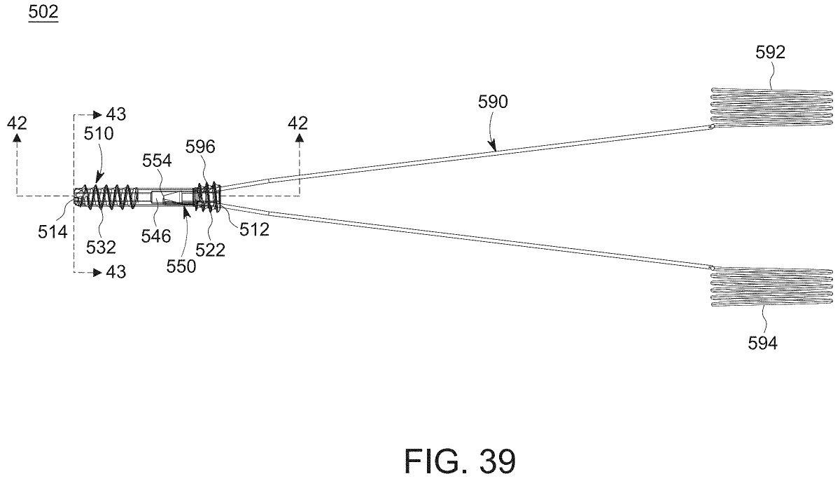

In addition, as shown in FIGS. 54-55, 57, 59-60, and 62-63, the head 556 may also include a first channel or recess 574 and a second channel or recess 576. The first channel 574 may extend, for example, into the head 556 between the first arm 558, including the first projection 562, and the second arm 560, including the second projection 564. The second channel 576 may extend, for example, into the head 556 between the first arm 558, including the first projection 562, and the second arm 560, including the second projection 564. The first channel 574 may be positioned on a side of the head 556 opposite the second channel 576. The first and second channels 574, 576 may extend from the exterior surface into the bridge 566. Thus, the bridge 566 separates the first channel 574 from the second channel 576. In addition, the through hole 572 extends between the first channel 574 and the second channel 576 below the bridge 566. The first and second channels 574, 576 may extend into the head 556 a depth to allow for each portion of the suture 590 to be recessed within the head 556 of the anchor member 550 once inserted into the implant 510, as shown in FIGS. 39-43.

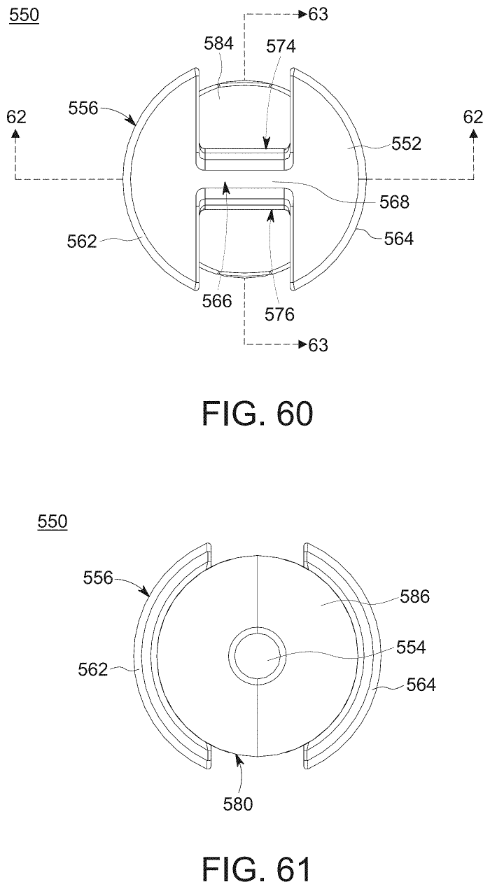

The shaft 580 may include a central portion 582, a coupling portion or tapered first end 584, and an insertion end or second end 586. The central portion 582 may be positioned between the coupling portion 584 and the insertion end 586. The coupling portion 584 is coupled to and extending from a bottom of the head 556 toward a second end 554 of the anchor member 550. The coupling portion 584 may have, for example, wider diameter at the end that couples with the head 556 than at the end that couples with a first end of the central portion 582. The central portion 582 may have a uniformly sized exterior surface as it extends between the coupling portion 584 and the insertion end 586. A second end of the central portion 582 is coupled to the insertion end 586. The insertion end 586 includes an engagement surface 588 where the coupling portion 582 couples to the insertion end 586. The engagement surface 588 may have a diameter wider than the diameter of the central portion 582 to form the engagement surface or top surface 588 of the insertion end 586. The insertion end 586 may, for example, taper from the engagement surface 588 to the second end 554 of the anchor member 550. The insertion end 586 may have, for example, a conical or truncated conical shape, as shown in FIGS. 54-59 and 61-63. As shown in FIGS. 60-61, the widest portion of the head 556 may have, for example, a diameter that is larger than the diameter of the engagement surface 588 of the insertion end 586.

The suture anchor, guide thread, suture, or surgical suture 590 is shown in FIGS. 34-43. The suture anchor 590 may include a first end 592, a second end 594, and the anchored portion 596 positioned between the first end 592 and the second end 594. As shown in FIGS. 34-36 and 42-43, the suture 590 may be inserted into the through hole 572 of the anchor member 550, such that the anchored portion 596 is positioned within the through hole 572 and the strands extending from the anchored portion 596 are positioned within the first and second channels 574, 576. A first strand of the strands extends to the first end 592 of the suture 590 and a second strand of the strands extends to the second end 594 of the suture 590. The anchored portion 596 may be positioned, for example, anywhere along the length of the suture anchor 590 between the first end 592 and the second end 594. The suture anchor 590 may be, for example, preloaded into the anchor member 550 before the surgical procedure or loaded into the anchor member 550 during the surgical procedure. The suture 590 may be loaded into the anchor member 550 using a suture threader 650, as described in greater detail below.

The suture 590 may be preloaded by, for example, inserting an end 592, 594 through the through hole 572 of the anchor member 550. The suture 590 may be loaded using the suture threader 650. After the suture 590 is loaded into the anchor member 550, the suture threader 650 may be removed and the anchor member 550 may be coupled to the inserter 600. The anchor member 550 may be coupled to the inserter 600 by aligning the end of the inserter 600 with the channels 574, 576 of the anchor member 550. Once the suture 590 is inserted through the through hole 572 and the inserter 600 is engaged with the anchor member 550, the first end 592 of the suture 590 may be coupled to a first arm 616 of the inserter 600 and the second end 594 of the suture 590 may be coupled to a second arm 622 of the inserter 600.

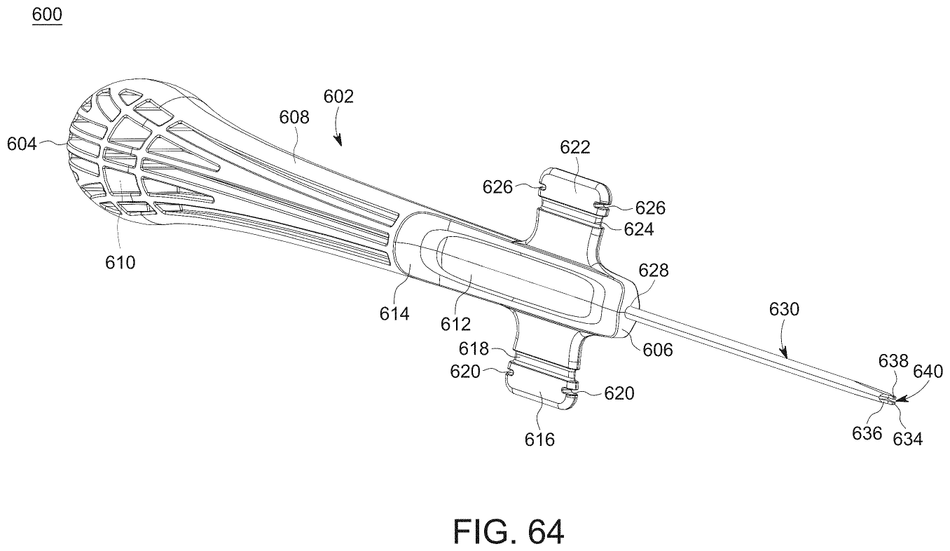

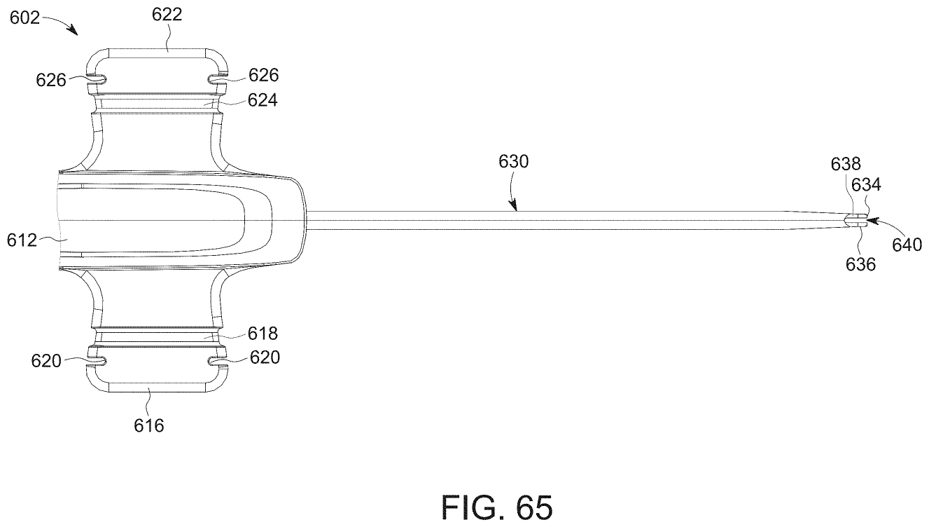

As shown in FIGS. 34-38 and 64-65, the inserter 600 may include a handle 602 and a drive member or insertion member 630. The handle 602 may include a first end 604 and a second end 606. The handle 602 may also include a first portion 608 extending from the first end 604 toward the second end 606 and a second portion 612 extending from the second end 606 toward the first end 604. The first portion 608 may be coupled to the second portion 612 at a coupling region or tapered region 614. The first portion 608 may also include, for example, a plurality of recesses 610 of various shapes and sizes along the length of the first portion 608. The plurality of recesses 610 may, for example, assist with grip of the handle 602 by the user. The first portion 608 may have, for example, heights extending between a top surface and a bottom surface of the handle 602 that are larger than the heights of the second portion 612.

With continued reference to FIGS. 34-38 and 64-65, the second portion 612 may include a first arm 616 extending away from a first side of the second portion 612 and a second arm 622 extending away from a second side of the second portion 612. The first arm 616 may extend away from the second portion 612 on a side opposite the second arm 622. The first and second arms 616, 622 may also extend away from the second portion 612 perpendicular to a longitudinal axis of the handle 602 extending between the first end 604 and the second end 606. The first and second arms 616, 622 may have, for example, a square or rectangular shape with curved, arced or rounded corners. The first arm 616 may include a first groove 618 inset into the first arm 616 in a longitudinal direction of the handle 602. The first groove 618 may be, for example, sized and shaped to receive at least one strand of the suture 590. The at least one strand of the suture 590 may be, for example, multiple wraps of the suture 590, depending on the length of the suture 590, to secure the suture in place during insertion of the anchor member 550. The first arm 616 may also include at least one first securement recess 620. The at least one first securement recess 620 may be positioned between the first groove 618 and a free end of the first arm 616. The at least one first securement recess 620 may be, for example, two first securement recesses 620. The at least one first securement recess 620 may extend into the first arm 616 in a direction along the longitudinal axis of the handle 602. The at least one first securement recess 620 may be, for example, sized and shaped to receive a first end 592 of the suture 590 to secure the suture 590 to the first arm 616 of the handle 602.

As also shown in FIGS. 34-38 and 64-65, the second arm 622 may include a second groove 624 inset into the second arm 622 in a longitudinal direction of the handle 602. The second groove 624 may be, for example, sized and shaped to receive at least one strand of the suture 590. The at least one strand of the suture 590 may be, for example, multiple wraps of the suture 590, depending on the length of the suture 590, to secure the suture in place during insertion of the anchor member 550. The second arm 622 may also include at least one second securement recess 626. The at least one second securement recess 626 may be positioned between the second groove 624 and a free end of the second arm 622. The at least one second securement recess 626 may be, for example, two second securement recesses 626. The at least one second securement recess 626 may extend into the second arm 622 in a direction along the longitudinal axis of the handle 602. The at least one second securement recess 626 may be, for example, sized and shaped to receive a second end 594 of the suture 590 to secure the suture 590 to the second arm 622 of the handle 602.

The handle 602 further includes an opening 628 extending into the second portion 612 of the handle 602 from a second end 606 toward the first end 604, as shown in FIGS. 34, 37, and 64. The opening 628 may extend along a length of the handle 602 between the first end 604 and the second end 606, but does not extend entirely between the first end 604 and the second end 606. The opening 628 is sized and shaped to receive a first end 632 and a portion of the drive member 630 extending from the first end 632. The second end 634 of the drive member 630 is positioned opposite the first end 632. The second end 634 may include a first projection or first engagement member 636, a second projection or second engagement member 638, and a channel 640 positioned between the first projection 636 and the second projection 638. The first projection 636 may be positioned on a first side of the drive member 630 and the second projection 638 may be positioned on a second side of the drive member 630. Each projection 636, 638 may be sized and shaped to engage the channels 574, 576. The projections 636, 638 may, for example, extend to contact a portion of the suture 590 extending through the through hole 572 of the anchor member 550. The inserter 600 may be coupled to the anchor member 550 before or after insertion of the suture 590 into the anchor member 550. Once the projections 636, 638 are positioned within the channels 574, 576 of the anchor member 550, the anchor member 550 may be inserted into the implant 510.

Referring now to FIGS. 34-38, the suture threader 650 is shown. The suture threader 650 includes a first side 652 and a second side 654 opposite the first side 652. The first side 652 may be, for example, arced or curved between a first end and a second end. The first side 652 may, for example, have a semicircular shape or the like to provide an ergonomic grip during insertion of the suture 590 into the anchor member 550. The second side 654 may be, for example, flat, planar or otherwise smooth for contacting the drive member 630 and anchor member 550 during insertion of the suture 590. The second side 654 may also include a threading member 656 positioned near a second end of the second side 654. The threading member 656 may include a portion for receiving the suture 590 and a tip for inserting the threading member 656 coupled to the suture 590 through the through hole 572 of the anchor member 550. In addition, the threading member 656 may include at least two legs 658, 660, 662 extending away from the second side 654 between the threading member 656 and the first end. In the depicted embodiment three legs 658, 660, 662 are shown. The three legs 658, 660, 662 include a first leg 658, a second leg 660, and a third leg 662. The first leg 658 is positioned near the second end of the second side 654, between the threading member 656 and the first end of the suture threader 650, and adjacent to a bottom of the suture threader 650. The second leg 660 is positioned near the first end of the second side 654, between the first leg 658 and the third leg 662, and adjacent to a top of the suture threader 650. The third leg 662 is positioned near the first end of the second side 654, between the second leg 660 and the first end of the second side 654, and adjacent to the bottom of the suture threader 650. Each leg 658, 660, 662 may be, for example, shaped to receive and removably couple to the drive member 630. Each leg 658, 660, 662 may, for example, deform as the drive member 630 is inserted into the suture threader 650 and then return to its original position.

A surgical method of using the orthopaedic compression anchor system 500 is shown in FIG. 66. The surgical method may include obtaining an orthopaedic compression anchor 700. In addition, the method may include preparing the bones surrounding the fracture site as appropriate for the specific fracture or fusion 710. Once the bones are prepared, the fracture can be stabilized 720 and the length of the screw required should be measured 730. Next, an opening may optionally be predrilled into cortical bone 735. A fixation device, k-wire, guide wire, or the like may then be inserted across the fracture and an imagining device, such as a mini C-arm, may be used to verify the position of the k-wire 740. In another embodiment, the k-wire could be inserted before predrilling the bone. Next, the implant may be inserted across the fracture site and through the predrilled opening, if made, to maintain compression across the fracture 750. The anchor member may then be preloaded with the suture, if the implant will also be used as an anchor for ligamentous, tendon, or other soft tissue repair 760. The anchor member may then be inserted into the implant until secured inside the implant 770. The anchor member may be inserted with a custom inserter tool. The custom inserter tool will be configured to manage the free ends of the suture. Thus, if using the inserter 600, the suture ends 592, 594 should be inserted through the through hole 572, and out of the anchor member 550 via the channels 574, 576, respectively. Once the anchor member is secured in the implant, the inserter may be pulled out and the sutures may be released 780. After the sutures are released, the sutures may be loaded into needles for performing the repair 790. The ligament, tendon and/or soft tissue repair may then be completed including closing any incisions made during the repair 800.

The surgical method may be used for articular fractures and other fractures to obtain compression across the fracture line while allowing for congruent cartilaginous surfaces to remain intact. The suture 590 coupled to the anchor member 550 and implant 510 allows for soft tissue repair in the area of the fracture without the need for a separate suture anchor. In one embodiment, the orthopaedic compression anchor 502 may be used for repairing a scaphoid fracture and associated soft tissue injuries. In other embodiments, the orthopaedic compression anchor 502 may be used for repairing other fractures of the bones of the hand, wrist, foot, ankle, and other like bones.

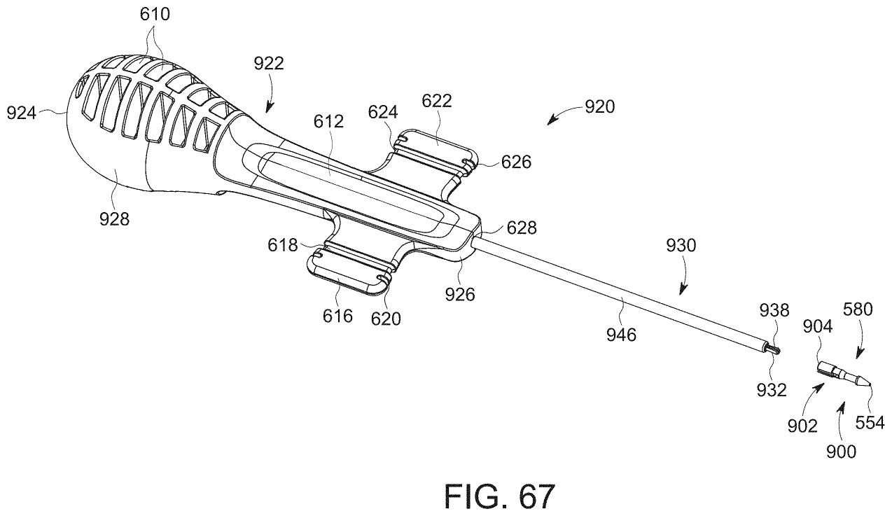

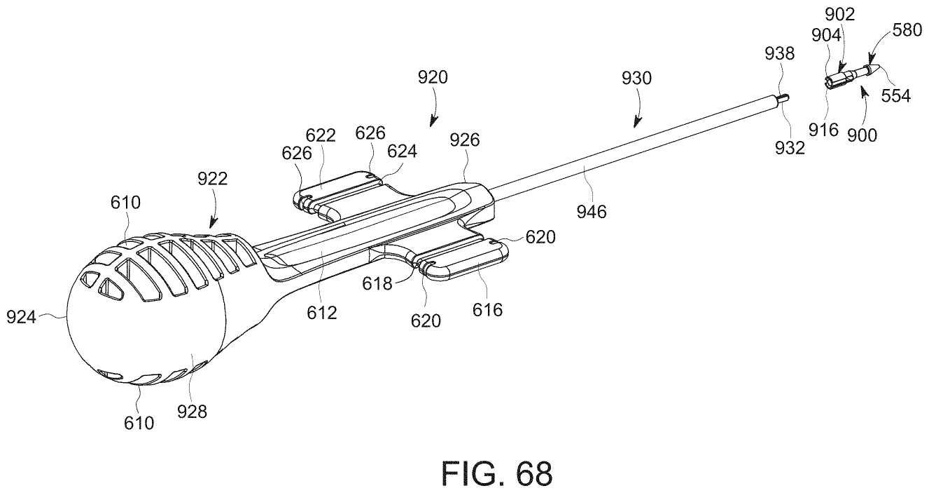

Referring now to FIGS. 67-70, another anchor member 900 and another inserter 920 with another handle 922 and another driver member or insertion member 920 are shown. The anchor member 900 includes a head 902 and a shaft 580. The head 902 includes a first end 904 and a second end 906. The head 902 may also include a first arm 908 and a second arm 910 extending from the first end 904 toward the second end 906 and the arms 908, 910 may extend out circumferentially from the head 902. The first arm 908 may be, for example, spaced apart from the second arm 910 by a first recess 912 on a first side and a second recess 914 on a second side. The first recess 912 may be, for example, spaced circumferentially around the head 902 from the second recess 914, such that the first recess 912 is positioned opposite the second recess 914. The recesses 912, 914 may extend into the head 902 from the exterior surface toward an inserter engagement opening 916. The inserter engagement opening 916 may extend from the first end 904 of the head 902 to the through hole 572. The opening 916 may be, for example, sized and shaped to receive a portion of the inserter 920. The shaft 580 is as described in greater detail above with reference to FIGS. 54-63 and which will not be described again here for brevity's sake.

The handle 922 of the inserter 920 includes a first end 924 and a second end 926 opposite the first end 924. The handle 922 includes a first portion 928 and a second portion 612. The first portion 928 extends from the first end 924 toward the second end 926. The first portion 928 also includes a plurality of recesses 610 extending into the first portion 928 of the handle 922. The recesses 610 may be, for example, various shapes and sizes as described in greater detail above and which will not be described again here for brevity's sake.

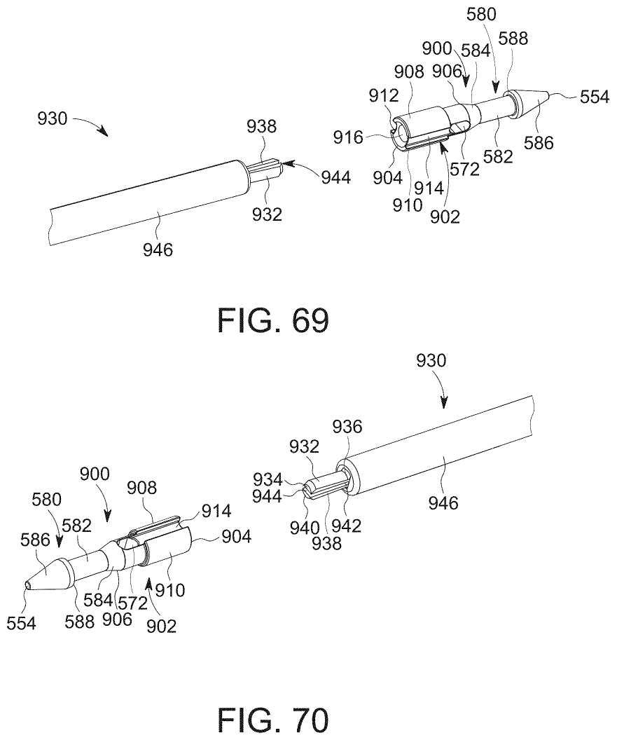

As shown in FIGS. 69-70, the driver member 930 may include a first arm 932 and a second arm 938 spaced apart from the first arm 932 by a channel 944. The first arm 932 may have a first end 934 and a second end 936. The second end 936 is coupled to and extends away from the base 946 of the driver member 930. The exterior surface of the first arm 932 may, for example, taper from the first end 934 to the second end 936. Thus, the first end 934 may be larger than the second end 936. The second arm 938 may have a first end 940 and a second end 942. The second end 942 is coupled to and extends away from the base 946 of the driver member 930. The exterior surface of the second arm 938 may, for example, taper from the first end 940 to the second end 942. Thus, the first end 940 may be larger than the second end 942. The channel 944 positioned between the first arm 932 and the second arm 938 allows for deformation of the first and second arms 932, 938 during insertion of the arms 932, 938 of the driver member 930. In addition, once inserted the arms 932, 938 exert a force on the sides of the opening 916 to retain the driver member 930 within the opening 916 of the anchor member 900 until after the anchor member 900 is inserted into an implant, such as, implant 510.

The anchor member 900 may be used with the surgical method described in FIG. 66.

As may be recognized by those of ordinary skill in the art based on the teachings herein, numerous changes and modifications may be made to the above-described and other embodiments of the present disclosure without departing from the scope of the disclosure. The devices, implants, and/or systems as disclosed in the specification, including the accompanying abstract and drawings, may be replaced by alternative component(s) or feature(s), such as those disclosed in another embodiment, which serve the same, equivalent or similar purpose as known by those skilled in the art to achieve the same, equivalent or similar results by such alternative component(s) or feature(s) to provide a similar function for the intended purpose. In addition, the devices, implants and systems may include more or fewer components or features than the embodiments as described and illustrated herein. For example, the components and features of the compression anchor systems of FIGS. 1-20, FIGS. 21-32, and FIGS. 34-65 may all be used interchangeably and in alternative combinations as would be modified or altered by one of skill in the art. Accordingly, this detailed description of the currently-preferred embodiments is to be taken in an illustrative, as opposed to limiting of the disclosure.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprise” (and any form of comprise, such as “comprises” and “comprising”), “have” (and any form of have, such as “has”, and “having”), “include” (and any form of include, such as “includes” and “including”), and “contain” (and any form of contain, such as “contains” and “containing”) are open-ended linking verbs. As a result, a method or device that “comprises,” “has,” “includes,” or “contains” one or more steps or elements possesses those one or more steps or elements, but is not limited to possessing only those one or more steps or elements. Likewise, a step of a method or an element of a device that “comprises,” “has,” “includes,” or “contains” one or more features possesses those one or more features, but is not limited to possessing only those one or more features. Furthermore, a device or structure that is configured in a certain way is configured in at least that way, but may also be configured in ways that are not listed.

The invention has been described with reference to the preferred embodiments. It will be understood that the architectural and operational embodiments described herein are exemplary of a plurality of possible arrangements to provide the same general features, characteristics, and general system operation. Modifications and alterations will occur to others upon a reading and understanding of the preceding detailed description. It is intended that the invention be construed as including all such modifications and alterations.

Having thus described the preferred embodiments, the invention is now claimed to be:

1. An anchor system, comprising:
an implant; and
a suture coupled to at least a portion of the implant.

2. The anchor system of claim 1, wherein the suture is at least one of directly coupled to at least a portion of the implant and indirectly coupled to at least a portion of the implant.

3. The anchor system of claim 1, further comprising:
an anchor member with at least one opening for receiving the suture;
wherein the anchor member is received within a through hole.

4. The anchor system of claim 3, wherein the implant further comprises:
a head at a first end of the implant;
a shaft coupled to and extending from the head to a second end of the implant; and
wherein the through hole extends through the implant from the first end to the second end.

5. The anchor system of claim 4, wherein the head of the implant further comprises:
a first threaded section positioned on and extending away from at least a portion of an exterior surface of the head; and
a drive opening extending into the first end of the head, wherein the drive opening overlaps with at least a portion of the through hole.

6. The anchor system of claim 5, wherein the shaft of the implant further comprises:
an interior surface positioned along a portion of the through hole, wherein the anchor member is positioned within the through hole along the portions where the drive opening and the interior surface extend;
an opening extending into a portion of the shaft from an exterior surface into the interior surface of the through hole; and
an engagement member having a first end and a second end and positioned within the opening, wherein a second end of the engagement member extends into the through hole of the implant.

7. The anchor system of claim 6, wherein the anchor member comprises:
a head extending from a first end of the anchor member toward a second end; and
a shaft extending from an end of the head to the second end of the anchor member.

8. The anchor system of claim 7, wherein the head of the anchor member comprises:
a first arm;
a second arm spaced apart from and positioned opposite the first arm;
a bridge connecting the first arm and the second arm along at least a portion of the first arm and the second arm; wherein the head of the anchor member further comprises:
a through hole extending through the head between the first arm and the second arm, wherein the through hole is positioned inferior to the bridge; and
a first channel extending into the head between the first arm and the second arm on a first side, wherein the first channel extends into the head to a first side of the bridge; and
a second channel extending into the head between the first arm and the second arm on a second side, wherein the second channel extends into the head to a second side of the bridge; wherein the first channel and the second channel are aligned with the through hole for receiving a portion of the suture.

9. The anchor system of claim 8, wherein the shaft of the anchor member comprises:
a coupling portion coupled to and extending from an end of the head;
a central portion coupled to and extending from the coupling portion; and
an insertion end coupled to and extending from the central portion.

10. The anchor system of claim 9, wherein the insertion end comprises:
an engagement surface positioned where the central portion couples to the insertion end, wherein the engagement surface extends beyond an exterior surface of the central portion.

11. The anchor system of claim 10, wherein the coupling portion is tapered from the head to the central portion, and wherein the insertion end tapers from the central portion to the second end.

12. The anchor system of claim 11, wherein a first portion of the suture extends through the through hole of the head of the anchor member, a second portion of the suture is positioned within the first channel of the head of the anchor member, and a third portion of the suture is positioned within the second channel of the head of the anchor member.

13. The anchor system of claim 12, further comprising:
an inserter, wherein the inserter comprises:
a handle extending from a first end of the inserter toward a second end, wherein the handle comprises:
a first portion extending from a first end toward a second end; and
a second portion extending from the first portion to the second end; and
a drive member inserted within an opening in the handle and extending from the handle to the second end of the inserter.

14. The anchor system of claim 13, wherein the second portion comprises:
a first arm extending from a first side of the second portion, wherein the first arm comprises:
a first groove inset into the first arm for receiving a first portion of the suture; and
at least one first securement recess for securing a first end of the suture;
a second arm extending from a second side of the second portion, wherein the first arm is positioned opposite the second arm, wherein the second arm comprises:
a second groove inset into the second arm for receiving a second portion of the suture; and
at least one second securement recess for securing a second end of the suture.

15. The anchor system of claim 14, wherein the drive member comprises:
a first end for insertion into the opening of the handle; and
a second end for engaging the anchor member, wherein the second end comprises;
a first projection extending from a first side of the drive member;
a second projection extending from a second side of the drive member; and
a channel positioned between the first projection and the second projection;
wherein the first projection and the second projection are tapered as the first and second projections extend from the drive member to the second end; and wherein the first projection is received within the first channel of the anchor member and the second projection is received within the second channel of the anchor member.

16. The anchor member of claim 15, further comprising:
a suture threader removably engaging the drive member of the inserter.

17. A surgical method of inserting a compression anchor, comprises:
obtaining a compression anchor;
preparing bones around a fracture site;
inserting an implant of the compression anchor across the fracture site;
performing a soft tissue repair with at least one suture of the compression anchor; and
completing the surgical procedure.

18. The surgical method of claim 17, wherein preparing the bones around the fracture site comprises:
stabilizing the fracture;
measuring a length of the implant needed;
inserting a fixation device across the fracture; and
using imaging to verify the position of the fixation device.

19. The surgical method of claim 18, further comprising:
preloading an anchor member of the compression anchor with the at least one suture;
coupling an inserter with the anchor member;
securing the at least one suture to the inserter;
inserting the anchor member and coupled at least one suture into the implant;
removing the inserter from the anchor member;
releasing the sutures from the inserter; and
performing a soft tissue repair with the at least one suture.

20. A method of assembling a compression anchor, comprising:
obtaining an implant, an anchor member, and a suture anchor;
obtaining a suture with a first end, a second end, and at least one anchor portion;
positioning the first end of the suture outside the implant;
positioning an anchor portion of the at least one anchor portion in a through hole of the anchor member;
positioning the second end of the suture outside the implant; and
inserting the coupled anchor member and suture into a through hole in the implant until a portion of the implant engages a portion of the anchor member.
